(19) United States
(12) Patent Application Publication
(10) Pub. No.: US 2022/0267390 A1
(43) Pub. Date: Aug. 25, 2022

(54) MPS MODIFIED PEPTIDES AND USE THEREOF

(71) Applicant: The Regents of the University of California, Oakland, CA (US)

(72) Inventors: Reen Wu, Davis, CA (US); Ching-Hsien Chen, Davis, CA (US); David C. Yang, Davis, CA (US)

(21) Appl. No.: 17/611,511

(22) PCT Filed: May 15, 2020
(86) PCT No.: PCT/US2020/033188

§ 371 Date: Nov. 15, 2021

Related U.S. Application Data
(60) Provisional application No. 62/849,637, filed on May 17, 2019.

(51) Int. Cl. C07K 14/47; C12N 15/86; A61P 11/00; A61K 31/496; A61K 31/4418; A61K 38/17; A61K 45/06

(52) U.S. Cl. CPC C07K 14/4703; C12N 15/86; A61P 11/00; A61K 31/496; C12N 2750/14171; A61K 38/1709; A61K 45/06; C12N 2750/14143; A61K 31/4418

(57) ABSTRACT

This disclosure provides an isolated polypeptide therapeutics, polynucleotides encoding the polypeptides and antibodies that bind to the polypeptides are also provided. Therapeutic and diagnostic uses are further provided.

### CROSS-REFERENCE TO RELATED APPLICATIONS

This application claims priority under 35 U.S.C. 119(e) to U.S. Provisional Application No. 62/849,637, filed May 17, 2019, the contents of which are hereby incorporated by reference into the present application in their entireties.

### STATEMENT OF GOVERNMENT SUPPORT

This disclosure was made with government support under the Grant No. R01HL077902, awarded by the NIH/NHLBI. Accordingly, the U.S. Government has certain rights to the disclosure.

### SEQUENCE LISTING

The instant application contains a Sequence Listing and is hereby incorporated by reference in its entirety. An ASCII copy, created on May 14, 2020, is named 060933-0740_SL.txt and is 41,917 bytes in size.

### BACKGROUND

The identification of MARCKS protein dates back to 1982 when it was found that an 87 kDa acidic protein in rat brain nerve endings could be regulated by calcium and calmodulin through the activation of PKC (Wu, W.C. et al. (1982) Proc. Natl. Acad. Sci. USA 79(17):5249-5253). Subsequently, the protein was officially named [symbol] yristoylated [symbol] lanine- [symbol] iic [symbol] [symbol] inase [symbol] ubstrate (MARCKS or MARKS) (Albert, K. A. et al. (1986) Proc. Natl. Acad. Sci. USA 83(9):2822-2826). MARCKS is ubiquitously expressed in various species and tissues (Albert, K. A. et al. (1987) Proc. Natl. Acad. Sci. USA 84(20):7046-7050; Stumpo, D. J. et al. (1989) Proc. Natl. Acad. Sci. USA 86(11):4012-4016), while the other MARCKS family member, MARCKS-related protein (MRP, also known as MacMARCKS, F52 or MLP), a 20 kDa protein is highly expressed in brain, reproductive tissues and macrophage (Aderem, A. (1992) Trend. Biochem. Sci. 17(10):438-443; Blacksher, P. J. (1993) J. Biol. Chem. 268:1501-1504). MRP, similar to MARCKS also contains the same three evolutionarily conserved domains; N-terminus myristoylation domain, multiple homology 2 (M12) domain, and the effector domain (ED). The MH12 domain of unknown function resembles the cytoplasmic tail of the cation-independent mannose-6-phosphate receptor. Protein phosphorylation occurs at Ser^{159/163} of ED domain. The corporation between the N-terminus (myristoylated) and the ED (phosphorylated or not phosphorylated) is essential for controlling the association of these molecules with membranes.

This disclosure provides an isolated polypeptide or an MPS polypeptide comprising, or alternatively consisting essentially of, or yet consisting of an amino acid sequence selected from the group of SEQ ID NOs: 45 or 40-59, or an equivalent of each thereof. In one aspect, an equivalent of the isolated polypeptide comprises or alternatively consists essentially of, or yet consists of a polypeptide having at least 80% sequence identity to the isolated polypeptide or a polypeptide encoded by a polynucleotide that hybridizes to an isolated polynucleotide that encodes the isolated polypeptide or its complement or a polypeptide encoded by a polynucleotide that having at least 80% sequence identity to the polynucleotide that encodes an amino acid sequence selected from the group of SEQ ID Nos. 45 or 40-59. In one aspect, the equivalent polypeptide has at least 80% sequence identity to the isolated polypeptide or a polypeptide encoded by a polynucleotide that hybridizes to an isolated polynucleotide that encodes the isolated polypeptide or its complement or a polypeptide encoded by a polynucleotide that having at least 80% sequence identity to the polynucleotide that encodes an amino acid sequence and not substituted at the residues that are D-amino acids, and they retain D-amino acids.

In another aspect, the isolated polypeptide or its equivalent comprises, or alternatively consists essentially of, or yet consists of no more than 51 amino acids. In another aspect, the isolated polypeptide or its equivalent comprises, or alternatively consists essentially of, or yet consists of no more than 35 amino acids. In a further aspect, the isolated polypeptide or its equivalent further comprises, or alternatively consists essentially of, or yet consists of one or more of: an operatively linked amino acid sequence to facilitate entry of the isolated polypeptide into the cell; a targeting polypeptide or a polypeptide that confers stability to the polypeptide.

Further provided are isolated polynucleotides encoding the polypeptides described above, complements of the polynucleotides and equivalents of each thereof.

Also disclosed is a vector comprising, or alternatively consisting essentially of, or yet further consisting of one or more of the isolated polynucleotide of this disclosure and optionally regulatory sequences operatively linked to the isolated polynucleotide for replication and/or expression. In one particular aspect, the vector is an AAV vector (adeno-associated viral vector). Further disclosed herein is a host cell further comprising the one or more of the isolated polypeptide, the isolated polynucleotide, or the vector of this disclosure. The host cell is a eukaryotic cell or a prokaryotic cell.

Compositions comprising, or alternatively consisting essentially of, or yet further consisting of a carrier and one or more of the isolated polypeptide, the isolated polynucleotide, the vector or the host cell of this disclosure are provided herein. In one aspect, the carrier is a pharmaceutically acceptable carrier. In a further aspect, the compositions of this disclosure can further comprise, or alternatively consist essentially of, or yet further consist of an additional therapeutic drug depending on the intended use, e g., a chemotherapeutic agent or drug, or an anti-fibrotic agent or drug. Non-limiting examples of an anti-fibrotic agent or drug include pirfenidone and nintedanib. Non-limiting examples of a chemotherapeutic agent or drug include for example such as a tyrosine kinase inhibitor (TKI) such as VEGFR, a platinum-based drug such as cisplatin, or a drug or agent that targets EGFR.

The compositions as disclosed herein are useful diagnostically, therapeutically and for screening methods as disclosed herein. They also can be used in the preparation of a medicament. Additionally, an additional agent or drug can be combined with the compositions within the same formulation or contained within a separate formulation but administered in combination to a subject in need thereof under appropriate conditions and in therapeutically effective amounts. The medicaments can be in the therapeutic methods as described herein.

Methods of treating disease or disease symptoms associated with fibrosis in a subject in need thereof, comprising, or alternatively consisting essentially of, or yet further consisting of administering to the subject an effective amount of one or more of the isolated polypeptide or the isolated polynucleotide of this disclosure are also provided. In one aspect, the disease or symptoms associated with fibrosis is selected from the group of: lung fibrosis, idiopathic pulmonary fibrosis, bleomycin-induced pulmonary fibrosis, kidney fibrosis, liver fibrosis, skin fibrosis, fibroblastic lesions, activated fibroblast proliferation, inflammation, or myofibroblast genesis. In a further aspect, the methods of treatment further comprise, or alternatively consist essentially of, or yet further consist of administering an effective amount of an anti-fibrotic agent or drug. Non-limiting examples of anti-fibrotic agent or drug include pirfenidone and nintedanib.

Also provided herein are methods for one or more of inhibiting cancer cell growth, treating cancer, inhibiting metastasis, inhibiting cancer stem cell growth, inhibiting tumor cell mobility, or restoring sensitivity of a resistant cancer cell to a chemotherapeutic agent, all in a subject in need thereof, by administering to the subject an effective amount of one or more of the isolated polypeptide or the isolated polynucleotide of this disclosure. In one aspect, the cancer cell or cancer is lymphoma, leukemia or a solid tumor. In another aspect, the solid tumor is a cancer of the type lung cancer, liver cancer, kidney cancer, brain cancer, colorectal cancer, pancreatic cancer, bone cancer, or throat cancer. In another aspect, the methods of treatment further comprise, or alternatively consist essentially of, or yet further consist of administering an effective amount of an anti-cancer drug or agent that may or may not be an MPS peptide or polynucleotide encoding the MPS peptide. In a further aspect, the methods of treatment further comprise, or alternatively consist essentially of, or yet further consist of administering an effective amount of a chemotherapeutic such as tyrosine kinase inhibitor, a platinum drug or an immunotherapeutic.

In one particular aspect, disclosed herein is a method for delivering a polypeptide of this disclosure across the blood brain barrier in a subject in need thereof comprising, or alternatively consisting essentially of, or yet further consisting of administering an effective amount of vector as disclosed above to the subject.

Administration can be local or systemic, e.g., topical or by inhalation therapy. Systemic administration can comprise of by a nebulizer, oral, intrathecal, topical, direct installation, sublingual, intravenous, intracranial, inhalation therapy, intranasal, vaginal or rectal administration.

Mammals such as an equine, murine, feline, canine, or human can be treated by the methods of this disclosure.

Kits are also provided. The kits comprise, or alternatively consist essentially of, or yet further consist of one or more of: an isolated polypeptide, an isolated polynucleotide, a vector, the cell or a composition of this disclosure and instructions for use. In one aspect, the instructions recite the methods of using the isolated polypeptide, the isolated polynucleotide, the cell, the vector, or the composition disclosed herein.

### BRIEF DESCRIPTION OF THE DRAWINGS

FIGS. 1A and 1B: Upregulation of MARCKS in IPF fibroblasts. (FIG. 1A) Graphical representation of computational analysis using IPF fibroblast profiling datasets (GSE21369 and GSE2052). (FIG. 1B) Normalized expression of MARCKS in IPF versus normal fibroblasts in GSE2052. FIGS. 1C and 1D: Upregulated MARCKS and PIP3 levels in idiopathic pulmonary fibrosis (IPF). (FIG. 1C). Expression levels of MARCKS and PIP3 in three normal fibroblasts and three-IPF fibroblast cells as stained with anti-MARCKS and anti-PIP3 antibodies. Tritc-conjugated MARCKS, FITC-conjugated PIP3 and nucleus counterstained DAPI were visualized under a confocal laser-scanning microscope. Scale bar: 10 μm. (FIG. 1D) Quantified cellular fluorescence levels for MARCKS and PIP3. Corrected total cell fluorescence for signal intensity of PIP3 and MARCKS were quantified and calculated with ImageJ.

FIG. 2: Unregulated MARCKS in IPF fibroblasts. Left, Expression of MARCKS mRNA as measured by real-time RT_{-q}PCR (n=5; *p<0.05 vs. Normal-1). Right, MARCKS protein and its phosphorylation were confirmed by Western blotting.

FIG. 3: Effects of MARCKS knockdown on primary IPF fibroblasts cell motility as determined by a wound healing assay (n=3).

FIGS. 4A-4B: MARCKS inhibition using MPS peptide reduced primary IPF fibroblast cell motility (FIG. 4A) and colony-forming ability (FIG. 4B) n=4; *, p<0.05.

FIG. 5: Representative images by using anti-pSer159/163 MARCKS antibody in normal lung tissue (left, n=10) and IPF specimens from patients without (middle, n=15) or with nintedanib treatment (right, n=3).

FIG. 6: Left, representative immunofluorescence images of phospho-MARCKS (light gray) and α-SMA (dark gray) in saline- or bleomycin-treated lunch tissues. DAPI (blue color): nucleus stains, Right, quantification of positive staining cells (n=3).

FIGS. 7A-7B: (FIG. 7A) Western blots analysis of phospho-MARCKS, phospho-AKT and α-SMA expression in lung fibroblast cells isolated from saline- or bleomycin-treated mice after 48 hours of treatment with control or MPS peptides (100 μM). (FIG. 7B) Effect of MPS peptide on cell viability of lung fibroblasts isolated from saline- (mFb-Saline) or bleomycin-treated (mFb-Bleomycin) mice (n=4; *, p<0.05).

FIG. 8: Body weight of mice in bleomycin-induced pulmonary fibrosis and MPS treatment.

FIG. 9: Left, representative Masson trichrome-stained sections of mouse lung with various treatments. Magnification: 4× (top) and 20× (bottom). Right, semiquantitative fibrosis score from Masson trichrome-stained sections of mouse lung. Fibrosis score is expressed as the percentage of positive staining area per high-powered field. Analysis of 6 to 12 high-powered fields per lung was performed with ImageJ software. *, p<0.05 (n=5).

FIGS. 10A-10C: (FIG. 10A) The PIP2-binding motif (SEQ ID NO: 12) on the [symbol] hosphorylation [symbol] ite [symbol] omain (PSD) of MARCKS. FIG. 10A discloses the MH domain as SEQ ID NO: 86). (FIG. 10B) Biolayer interferometry analysis of the binding of the MPS peptide to biotin-labeled PIP2. (FIG. 10C) PIP3 levels in IPF fibroblast cells with PBS or MPS treatment. * p<0.05 versus PBS (n=3).

FIGS. 11A-11B: (FIG. 11A) Western blot analysis of α-SMA and phospho-AKT in primary IPF fibroblasts with nintedanib (1000 nM) and/or MPS (100 μM) for 48 hours. (FIG. 11B) A proposed model of activating the PI3K/AKT pathway after nintedanib treatment. An arrow: a direct interaction.

FIGS. 12A-12E: (FIGS. 12A-12B) Combinatorial effect of MPS peptide with nintedanib on fibroblasts isolated from two IPF patients. Cells were treated with various closes of nintedanib (62.5-2000 nM) and/or MPS peptide (6.25-200 μM) for 72 hours, respectively. After single (lined) or combined (lined) treatment, cell viability was determined by MTT assays. (FIG. 12C) The Chou and Talalay CI (combination index) method was utilized to evaluate the therapeutic interactions between nintedanib and MPS peptide using the Calcusyn software. Gray line, additive effect of the combination of MPS peptide and the drug is represented at CI=1. (FIG. 12D) Cells were individually treated with 1 μM nintedanib, 100 μM MPS peptide or combinations of 1 μM nintedanib and 100 μM MPS peptide. After 48 hours, cell viability was determined by the trypan blue exclusion assay (n+3; *, p<0.05). (FIG. 12E) shows selected polypeptides and their corresponding sequence ID number.

FIG. 13: The table shows the sequences of the MPS derivatives (SEQ ID NOS: 48-54, 40-42, 45 and 47, respectively, in order of appearance). IC₅₀ (half maximal inhibitory concentration; μM) values in lung cancer cells. FIG. 13 also shows a CLUSTAL O (1.2.4) multiple sequence alignment for various MPS-related peptides. The residues marked in red/bold are D-isoforms of amino acids (SEQ ID NOS: 57, 48-54, 40-42 45 and 47 in order of appearance).

FIG. 14: Comparison of MPS-12042 (SEQ ID NO: 45) versus know tyrosine kinase inhibitor (TKLs) on the treatment of IPF fibroblast cells. Both normal and IPF lung fibroblast cells were treated with various drugs. After 72 hours, cells were subjected to MTT assays and IC50 for each drug was determined.

FIG. 15: Left, RNA-seq of oncosphere derived from LG704 showed 325 genes significantly altered by MPS treatment. These genes were then analyzed with GSEA to determine which functional pathways were most affected by MARCKS. Right, Heat map of cancer-stemness markers associated with MARCKS activity.

FIG. 16: Top, phase contrast photomicrographs of oncospheres in non-adherence 3-D culture without (left) and with 10% CSE (right). Bottom, RT-qPCR analyses of mRNA expression in the above cells.

FIG. 17A-17B: (FIG. 17A) Sphere-forming assays for evaluating the effect of MARCKS phosphorylation on smoke-mediated stemness in cells with ectopic expression of wild type or PSD-mutated (S159/163A) MARCKS. (FIG. 17B) WB analyses of stemness markers in the above cells.

FIG. 18A-18C: (FIG. 18A) Sphere-forming assays for evaluating the inhibitory effect of the MPS peptide on smoke-mediated stemness. (FIG. 18B) Quantification of the number and size of oncospheres. (FIG. 18C) RT-qPCR analyses of mRNA expression in the above oncospheres.

FIG. 19 shows MARCKS mimetic peptide (MPS) targeting phospho-MARCKS, binds to PIP2, and inhibits production of PIP3. Multiple IPF lung fibroblast cells were treated with either PBS or 100 μM of MPS peptide for 12 hours and then subjected to immunocytochemistry using anti-PIP3 antibodies. Representative images are shown (n=3). Scale bar: 20 μm.

FIGS. 20A and 20B show suppressive effects of MPS peptide on pulmonary fibrosis in vivo. C57BL/6 mice were intraperitoneally given either PBS, nintedanib (28 mg/kg), MPS peptide (28 mg/kg) or MPS-12042 (7 mg/kg) at the dosage of every two days after 9 days of intratracheal exposure with one shot of saline or bleomycin (33 μg in 50 ml of saline, n=5). (FIG. 20A) Representative Masson trichrome and immunohistochemical staining of phospho-MARCKS (Ser159/163) and phospho-AKT (Ser473) (n=6). (FIG. 20B) Hydroxyproline level in the left lung of mice as described above was determined by a hydroxyproline ELISA assay (mean±SD, *p<0.05).

### DETAILED DESCRIPTION

Before the compositions and methods are described, it is to be understood that the disclosure is not limited to the particular methodologies, protocols, cell lines, assays, and reagents described, as these may vary. It is also to be understood that the terminology used herein is intended to describe particular embodiments of the present disclosure, and is in no way intended to limit the scope of the present disclosure as set forth in the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. Throughout this disclosure, various technical publication are identified by an Arabic number, with the full bibliographic citation provide immediately preceding the claims.

All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “binding” or “binds” as used herein are meant to include interactions between molecules that may be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “MARCKS” intends the protein that was officially named myristoylated alanine-rich C kinase substrate (MARCKS or MARKS) (Albert, K. A. et al. (1986) Proc. Natl. Acad. Sci. USA 83(9):2822-2826). MARCKS is ubiquitously expressed in various species and tissues (Albert, K. A. et al. (1987) Proc. Natl. Acad. Sci. USA 84(20):7046-7050; Stumpo, D. J. et al. (1989) Proc. Natl. Acad. Sci. USA 86(11):4012-4016), while the other MARCKS family member, MARCKS-related protein (MRP, also known as MacMARCKS, F52 or MLP), a 20 kDa protein is highly expressed in brain, reproductive tissues and macrophages (Aderem, A. (1992) Trend. Biochem. Sci. 17(10):438-443; Blackshear, P. J. (1993) J. Biol. Chem. 268:1501-1504). MRP, similar to MARCKS also contains the same three evolutionarily conserved domains; N-terminus myristoylation domain, multiple homology 2 (MH2) domain, and the effector domain (ED). The MH2 domain of unknown function resembles the cytoplasmic tail of the cation-independent mannose-6-phosphate receptor. Protein phosphorylation occurs at Ser159/163 of ED domain. The corporation between the N-terminus (myristoylated) and the ED (phosphorylated or not phosphorylated) is essential for controlling the association of these molecules with membranes.

In one aspect, the MPS polypeptide of this disclosure comprises, or alternatively consists essentially of, or yet consists of at least 6 amino acids and no more than 51 amino acids. In a further aspect, the polypeptide is at least 6 amino acids and no more than 51 amino acids, or alternatively at least 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids or alternatively or equivalents of each thereof. In one aspect, an equivalent is a polypeptide wherein one or more amino acids have been substituted with a conservative amino acid substitution.

The MPS polypeptides and equivalents thereof have the “biological activity” or the biological ability to: inhibit the expression of MARCKS for preventing, reducing, delaying, inhibiting or suppressing disease or disease symptoms associated with MARCKS phosphorylation and/or dissociation from the cell membrane and/or PIP2-sequestering effect, or PIP3 production, or activation of AKT, or inflammation, fibrosis, or activated fibroblast proliferation, or myofibroblast genesis and differentiation, or transforming growth factor-beta (TGF-β) signaling pathway, or cancer, tumor cell growth, solid tumor cell growth or metastasis, or cancer stem cell growth, cancer stemness, or tumor cell mobility; and optionally for promoting apoptosis, or restoring sensitivity of a resistant cancer cell to a chemotherapeutic. In one aspect, the MPS polypeptides and equivalents have the ability to prevent, reduce, delay, inhibit or suppress disease or disease symptoms associated with lung fibrosis, idiopathic pulmonary fibrosis, or smoking, bleomycin-induced pulmonary fibrosis, kidney fibrosis, liver fibrosis, skin fibrosis, fibroblastic lesions, activated fibroblast proliferation, inflammation, or myofibroblast genesis. In another aspect, the MPS polypeptides and equivalents have the ability to prevent, reduce, delay, inhibit or suppress disease or disease symptoms associated with lymphoma, leukemia or a solid tumor or cancer (carcinoma or sacrcoma). Non-limiting examples of solid tumor include cancer, lung cancer, kidney cancer, ovarian cancer, brain cancer, colorectal cancer, pancreatic cancer, bone cancer, or throat cancer. In one aspect, to “treat” excludes prevention or prophylaxis.

The term “polypeptide” is used interchangeably with the term “protein” and “peptide” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. In one aspect, the polypeptides contain unnatural or synthetic amino acids, including glycine and both the D and L optical isomers of naturally occurring amino acids, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component, that in one aspect, is a non-naturally occurring combination of polynucleotide and label. The term also refers to both double and single stranded molecules. Unless otherwise specified or required, any embodiment of this disclosure that is a polynucleotide encompasses both the double stranded form and each of two complementary single stranded forms known or predicted to make up the double stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Equivalent polynucleotides are those having the specified percent homology and/or encoding a polypeptide having the same or similar biological activity.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the RNA when a gene is transcribed or amino acid (e.g., peptide or polypeptide) generated when a gene is transcribed and translated.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

Applicant have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments.

A “gene delivery vehicle” is defined as any molecule that can carry inserted polynucleotides into a host cell. Examples of gene delivery vehicles are liposomes, micelles, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this disclosure can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a gene of interest. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Publication No. WO 95/27071. Ads do not require integration into the host cell genome. Recombinant Ad derived vectors, particularly those that reduce the potential for recombination and generation of wild-type virus, have also been constructed. See, International PCT Publication Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this disclosure. To enhance delivery to a cell, the nucleic acid or proteins of this disclosure can be conjugated to antibodies or binding fragments thereof which bind cell surface antigens. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this disclosure are other non-limiting techniques.

The terms “culture” or “culturing” refer to the in vitro propagation of cells, tissues, or organisms on or in media of various kinds. It is understood that the descendants of a cell grown in culture may not be completely identical (i.e., morphologically, genetically, or phenotypically) to the parent cell.

The term “antibody” herein is used in the broadest sense and specifically includes full-length monoclonal antibodies, polyclonal antibodies, multispecific antibodies (e.g., bispecific antibodies), and antibody fragments, so long as they exhibit the desired biological activity. As used herein the terms “antibodies” and “immunoglobulin” include antibodies or immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated CDRs or a functional paratope; chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin. a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are arrayed on a gene chip for determining the DNA sequence by the hybridization approach, such as that outlined in U.S. Pat. Nos. 6,025,136 and 6,018,041. The polynucleotides of this disclosure can be modified to probes, which in turn can be used for detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Cell,” “host cell” or “recombinant host cell” are terms used interchangeably herein. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, “treating” or “treatment” of a disease in a subject refers to (1) preventing the symptoms or disease from occurring in a subject that is predisposed or does not yet display symptoms of the disease; (2) inhibiting the disease or arresting its development or relapse; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, prevention or prophylaxis is excluded from the term “treatment.”

When the disease is cancer, the following clinical endpoints are non-limiting examples of treatment: reduction in tumor burden, slowing of tumor growth, longer overall survival, longer time to tumor progression, inhibition of metastasis, reduction in cancer stemness, or a reduction in metastasis of the tumor. In one aspect, treatment excludes prophylaxis. When the disease is fibrosis, the following clinical end points are non-limiting examples of treatment: reduction in fibrotic tissue, reduction in inflammation, reduction in fibroblastic lesions, reduction in activated fibroblast proliferation, reduction in myofibroblast genesis, reduction in rate of decline of Forced Vital Capacity (FVC), wherein FVC is the total amount of air exhaled during the lung function test, absolute and relative increases from baseline in FVC, absolute increase from baseline in FVC (% Predicted), increase in progression-free survival time, decrease from baseline in St George's Respiratory Questionnaire (SGRQ) total score, wherein SGRQ is a health-related quality of life questionnaire divided into 3 components: symptoms, activity and impact and the total score (summed weights) can range from 0 to 100 with a lower score denoting a better health status, and relative decrease from baseline in high resolution computerized tomography (HRCT) quantitative lung fibrosis (QLF) score, wherein the QLF score ranges from 0 to 100% and greater values represent a greater amount of lung fibrosis and are considered a worse health status. Non-limiting examples clinical end points for fibrosis treatment and tests that can be performed to measure said clinical end points are described in the following clinical trials: NCT03733444 (clinicaltrials.gov/ct2/show/NCT03733444) (last accessed on Jan. 9, 2019), NCT00287729 (clinicaltrials.gov/ct2/show/NCT00287729) (last accessed on Jan. 9, 2019), NCT00287716 (clinicaltrials.gov/ct2/show/NCT00287716) (last accessed on Jan. 9, 2019), NCT02503657 (clinicaltrials.gov/ct2/show/NCT02503657) (last accessed on Jan. 9, 2019), NCT00047645 (clinicaltrials.gov/ct2/show/NCT00047645) (last accessed on Jan. 9, 2019), NCT02802345 (clinicaltrials.gov/ct2/show/NCT02802345) (last accessed on Jan. 9, 2019), NCT01979952 (clinicaltrials.gov/ct2/show/NCT01979952) (last accessed on Jan. 9, 2019), NCT00650091 (clinicaltrials.gov/ct2/show/NCT00650091) (last accessed on Jan. 9, 2019), NCT01335464 (clinicaltrials.gov/ct2/show/NCT01335464) (last accessed on Jan. 9, 2019), NCT01335477 (clinicaltrials.gov/ct2/show/NCT01335477) (last accessed on Jan. 9, 2019), NCT01366209 (clinicaltrials.gov/ct2/show/NCT01366209) (last accessed on Jan. 9, 2019). Further non-limiting examples clinical endpoints for fibrosis treatment and tests that can be performed to measure said clinical end points are described in King et al, (2014) N Engl J Med. May 29; 370(22):2083-92 and Richeldi et al, (2014) N Engl J Med. May 29; 370(22):2071-82.

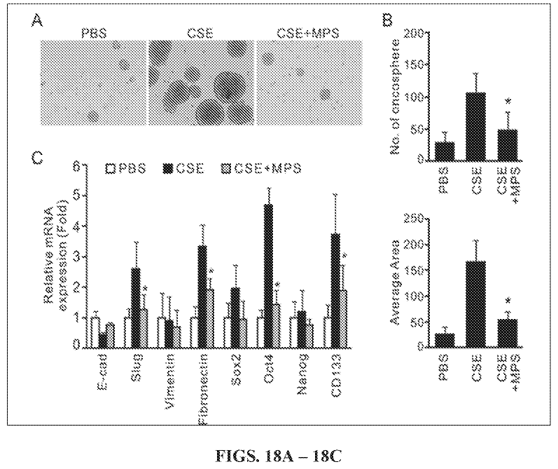

A “cancer stem cell” (“CSC”) intends a cell or a subpopulation of cells within tumors with capabilities of self-renewal, differentiation, and tumorigenicity when transplanted into an animal host. On the basis of the clinical evidence and experimental observations, CSCs appear to possess long-term clonal maintenance of cancer malignancy and survival even after many harsh therapy treatments. The gold standard for defining CSCs has been serial in vivo transplantation, but a number of cell surface markers such as Sox2, Slug, CD44, CD24, and CD133 have proved useful to study CSCs in patient specimens and experimental systems. A regulatory network consisting of microRNAs and Wnt/β-catenin, Notch, and Hedgehog signaling pathways controls the CSC properties. As used herein, one or more of these are intended as cancer stem cell markers. Additional markers are provided in FIGS. 15 and 17. Expression of these markers can be detected and monitored by methods known and described herein and in the art.

Sox2 (sex determining region Y (SRY)-box 2) intends the transcription factor that participates in maintaining self-renewal and pluripotency of embryonic stem cells. The expression of this protein is aberrant in various human malignancies, and has been reported to act as an oncogene in esophageal squamous cell carcinoma (SCC). It also has been reported to promote proilferation, migration and adhesion abilities of dental pulp stem cells (DPSCs). It is known to participate in Ewing's sarcoma cell proliferation, and its inactivation results in apoptosis and G1/S arrest, in a PI3K (phosphoinositide 3-kinase)/Akt pathway-mediated manner. Monoclonal antibodies to detect and monitor expression are commercially available, e.g., Sigma-Aldrich and Novus Biologicals (last accessed on May 6, 2020).

CD133 or CD133 antigen, also known as prominin-1, is a glycoprotein that in humans is encoded by the PROM1 gene. It is a member of pentaspan transmembrane glycoproteins, which specifically localize to cellular protrusions. Monoclonal antibodies to detect and monitor expression are commercially available, e.g., Abcam and ThermoFisher (last accessed on May 6, 2020).

Slug or (SNAI2) is a transcription factor and an inducer of the epithelial to mesenchymal transition which mediates cell migration during development and tumor invasion. Devendra et al. (2014) Stem Cells, December 32(12):3209-3218, 10.1002/stem.1809. Methods to detect and monitor expression are known in the art, e.g., Devendra et al. (2014), supra.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are known to be predisposed to the disease due to family history, being genetically predispose to developing the disease, or diagnosed with a disease or disorder that predisposes them to developing the disease to be treated.

“An effective amount” intends to indicate the amount of a compound or agent administered or delivered to the patient which is most likely to result in the desired response to treatment. The amount is empirically determined by the patient's clinical parameters including, but not limited to the stage of disease, age, gender, histology, sensitivity, toxicity and likelihood for tumor recurrence. In one aspect, an “effective amount” is a therapeutically effective amount.

As used herein, a “cancer” is a disease state characterized by the presence in a subject of cells demonstrating abnormal uncontrolled replication and may be used interchangeably with the term “tumor.” In some embodiments, the cancer is a solid tumor, lung cancer, liver cancer, kidney cancer, brain cancer, ovarian cancer, colorectal cancer, pancreatic cancer, bone cancer, throat cancer, lymphoma, or leukemia.

A “tumor” is an abnormal growth of tissue resulting from uncontrolled, progressive multiplication of cells and serving no physiological function. A “tumor” is also known as a neoplasm.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

“Having the same cancer” is used when comparing one patient to another or alternatively, one patient population to another patient population. For example, the two patients or patient populations will each have or be suffering from colon cancer.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, the disease being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application.

An agent of the present disclosure can be administered for therapy by any suitable route of administration. It will also be appreciated that the preferred route will vary with the condition and age of the recipient, and the disease being treated.

A tyrosine kinase inhibitor (“TKI”) is an agent (small molecule or biologic) that inhibits the action of tyrosine kinase in a cell. Tyrosine kinases are enzymes that are responsible for the activation of many proteins by signal transduction cascades. TKIs are typically used as anti-cancer drugs. Examples of tyrosine kinase inhibitors include, but are not limited to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib); FLT3 (Lestaurtinib); PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib). Small molecule TKIs are known in the art and listed at the web address comprising oncolink.org/treatment/article.cfm?id=452 (last accessed on Jul. 17, 2014).

PTK/ZK is a “small” molecule tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-1 (Flt-1), VEGFR-2 (KDR/Flk-1) and VEGFR-3 (Flt-4). The chemical names of PTK/ZK are 1-[4-Chloroanilino]-4-[4-pyridylmethyl] phthalazine Succinate or 1-Phthalazinamine, N-(4-chlorophenyl)-4-(4-pyridinylmethyl)-, butanedioate (1:1). Synonyms and analogs of PTK/ZK are known as Vatalanib, CGP79787D, PTK787/ZK 222584, CGP-79787, DE-00268, PTK-787, PTK-787A, VEGFR-TK inhibitor, ZK 222584 and ZK.

As used herein, the term “platinum-based drug” intends an anticancer drug that is a platinum based compound which is a subclass of DNA alkylating agents. Such agents are well known in the art and are used to treat a variety of cancers, such as, lung cancers, head and neck cancers, ovarian cancers, colorectal cancer and prostate cancer. Non-limiting examples of such agents include carboplatin, cisplatin, nedaplatin, oxaliplatin, triplatin tetranitrate, Satraplatin, Aroplatin, Lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004). “Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include without limitation cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

### Modes of Carrying Out the Disclosure

### Isolated Polypeptides and Compositions

This disclosure provides an isolated polypeptide or an MPS polypeptide comprising, or alternatively consisting essentially of, or yet consisting of an amino acid sequence selected from the group of: SEQ ID NOs 40-56, 58 and 59, or an equivalent of each thereof. In one aspect, the isolated polypeptides include substantially homologous and equivalent polypeptides. In one aspect, the isolated polypeptide of this disclosure comprises, or alternatively consists essentially of, or yet consists of no more than 51 amino acids. In another aspect, the isolated polypeptide of this disclosure comprises, or alternatively consists essentially of, or yet consists of no more than 35 amino acids. In a yet further aspect, the polypeptide is at least 6 amino acids and no more than 51 amino acids, or alternatively at least 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids or alternatively or equivalents of each thereof. In one aspect, an equivalent of the isolated polypeptide comprises or alternatively consists essentially of, or yet consists of a polypeptide having at least 80% sequence identity to the isolated polypeptide or a polypeptide encoded by a polynucleotide that hybridizes to an isolated polynucleotide that encodes the isolated polypeptide or its complement or a polypeptide encoded by a polynucleotide that having at least 80 sequence identity to the polynucleotide that encodes an amino acid sequence selected from the group of SEQ ID Nos. 40-56, 58 and 59.

High stringency hybridization conditions is generally performed at about 60° C. in about 1×SSC. Substantially homologous and equivalent polypeptides and substantially homologous and equivalent polynucleotides intend those having at least 80% homology, or alternatively at least 85% homology, or alternatively at least 90% homology, or alternatively, at least 95% homology or alternatively, at least 98% homology to those described above, each as determined using methods known to those skilled in the art and identified herein, when run under default parameters. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide, or alternatively identical nucleic acid sequence to the reference polynucleotide, when compared using sequence identity methods run under default conditions. In one specific aspect, they may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide, or alternatively identical nucleic acid sequence to the reference polynucleotide, when compared using sequence identity methods run under default conditions. In one aspect, an equivalent is a polypeptide wherein one or more amino acids have been substituted with a conservative amino acid substitution. In one aspect, the isolated polypeptide has at least one amino acid that is a modified, non-naturally occurring amino acid such as D-lysine.

In one aspect, the MPS polypeptide of this disclosure comprises, or alternatively consists essentially of, or yet consists of at least 6 amino acids and no more than 51 amino acids. In a further aspect, the polypeptide is at least 6 amino acids and no more than 51 amino acids, or alternatively at least 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids or alternatively or equivalents of each thereof. In one aspect, an equivalent is a polypeptide wherein one or more amino acids have been substituted with a conservative amino acid substitution. In a further aspect, myristic acid is conjugated or joined to the N-terminal amino acid, including equivalents thereof, e.g., wherein all serines are replaced by alanine. In one aspect, the isolated polypeptide has at least one amino acid that is a modified, non-naturally occurring amino acid such as D-lysine.

In one aspect, the isolated polypeptide as described above, have additional amino acids added onto the carboxyl-terminal end or amino-terminal end of the MPS and equivalents of each thereof, such that the length of the polypeptide comprises an additional at least 10 amino acids, or alternatively at least 15 amino acids, or alternatively at least 20 amino acids, or alternatively at least 25 amino acids, or alternatively at least 30 amino acids, or alternatively at least 35 amino acids or the addition of amino acids up to a total of 51 amino acids.

It is known to those skilled in the art that modifications can be made to any peptide to provide it with altered properties. Peptide fragments of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, R turns, R sheets, α-turns, and cyclic peptides can be generated. Generally, it is believed that α-helical secondary structure or random secondary structure is preferred. The disclosed polypeptides, in one aspect, contain unnatural amino acids.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobic, size, or charge. Methods used to determine the appropriate amino acid to be substituted and for which amino acid are known to one of skill in the art. Non-limiting examples include empirical substitution models as described by Layoff et al. (1978) In Atlas of Protein Sequence and Structure Vol. 5 suppl. 2 (ed. MR. Day off), pp. 345-352. National Biomedical Research Foundation, Washington D.C.; PAM matrices including Day off matrices (Layoff et al. (1978), supra, or JET matrices as described by Jones et al. (1992) Compute. Appl. Basic. 8:275-282 and Gannet et al. (1992) Science 256:1443-1145; the empirical model described by Adak and Hasegawa (1996) J. Mol. Evil. 42:459-468; the block substitution matrices (BLOSSOM) as described by Henrico and Henrico (1992) Proc. Natl. Acad. Sci. USA 89:10915-10919; Poisson models as described by Neil (1987) Molecular Evolutionary Genetics. Columbia University Press, New York.; and the Maximum Likelihood (ML) Method as described by Muller et al. (2002) Mol. Biol. Evil. 19:8-13.

Accordingly, in yet another aspect the isolated polypeptide or peptide fragment may comprise, or alternatively consisting essentially of, or yet further consisting of, a “an equivalent” or “biologically active” polypeptide encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions.

The isolated polypeptides or MPS polypeptides and equivalents have the ability to: inhibit the expression of MARCKS for preventing, reducing, delaying, inhibiting or suppressing disease or disease symptoms associated with MARCKS phosphorylation and/or dissociation from the cell membrane and/or PIP2-sequestering effect, or PIP3 production, or activation of AKT, or inflammation, fibrosis, or activated fibroblast proliferation, or myofibroblast genesis and differentiation, or transforming growth factor-beta (TGF-0) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic. In one aspect, the isolated polypeptides and equivalents have the ability to prevent, reduce, delay, inhibit or suppress disease or disease symptoms associated with lung fibrosis, idiopathic pulmonary fibrosis, or smoking, bleomycin-induced pulmonary fibrosis, kidney fibrosis, liver fibrosis, skin fibrosis, fibroblastic lesions, activated fibroblast proliferation, inflammation, or myofibroblast genesis. In another aspect, the isolated polypeptides and equivalents have the ability to prevent, reduce, delay, inhibit or suppress disease or disease symptoms associated with lymphoma, leukemia or a solid tumor. Non-limiting examples of solid tumor include cancer, kidney cancer, brain cancer, colorectal cancer, pancreatic cancer, bone cancer, or throat cancer.

The polypeptides are useful therapeutically to inhibit or suppress solid tumor growth such as cancer cell invasion, metastasis, migration and viability of cancer cells in vitro or in vivo. They also promote apoptosis and inhibit the growth of cancer stem cells (such as those expressing CD133+), malignant tumors and cancer cells, increase or induce cancer cell death.

In a further aspect, further provided is an isolated polypeptide further comprising, or alternatively consisting essentially of, or yet consisting of one or more of: an operatively linked amino acid sequence to facilitate entry of the isolated polypeptide into the cell; a targeting polypeptide or a polypeptide that confers stability to the polypeptide. Also provided is an isolated polypeptide wherein the amino acid sequence comprises, or alternatively consists essentially of, or alternatively consisting of an operatively linked polypeptide that targets the polypeptide to a specific cell type or stabilizes the polypeptide or yet further comprises a transduction domain for facilitated cell entry or tumor targeting domain and an MPS polypeptide as described herein.

Polypeptides comprising, or alternatively consisting essentially of, or yet further consisting of, the amino acid sequences of the disclosure can be prepared by expressing polynucleotides encoding the polypeptide sequences of this disclosure in an appropriate host cell. This can be accomplished by methods of recombinant DNA technology known to those skilled in the art. Accordingly, this disclosure also provides methods for recombinantly producing the polypeptides of this disclosure in a eukaryotic or prokaryotic host cell, which in one aspect is further isolated from the host cell. The proteins and peptide fragments of this disclosure also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

The protein and peptide fragments may be operatively linked to a transduction domain for facilitated cell entry. Protein transduction offers an alternative to gene therapy for the delivery of therapeutic proteins into target cells, and methods involving protein transduction are within the scope of the disclosure. Protein transduction is the internalization of proteins into a host cell from the external environment. The internalization process relies on a protein or peptide which is able to penetrate the cell membrane. To confer this ability on a normally non-transducing protein, the non-transducing protein can be fused to a transduction-mediating protein such as the antennapedia peptide, the HIV TAT protein transduction domain, or the herpes simplex virus VP22 protein. See Ford et al. (2001) Gene Ther. 8:1-4. As such the polypeptides of the disclosure can, for example, include modifications that can increase such attributes as stability, half-life, ability to enter cells and aid in administration, e.g., in vivo administration of the polypeptides of the disclosure. For example, polypeptides of the disclosure can comprise, or alternatively consisting essentially of, or yet further consisting of, a protein transduction domain of the HIV TAT protein as described in Schwarze et al. (1999) Science 285:1569-1572. In addition, or alternatively, the polypeptides include amino acid sequences that target the polypeptide to the cell or tissue to be treated and/or stabilizes the polypeptide.

In a further aspect, any of the proteins, peptides or polynucleotides of this disclosure can be combined with a detectable label such as a dye for ease of detection. Non-limiting examples of such include radioisotopes, fluorochromes, chemiluminescent compounds, dyes, and proteins, including enzymes.

The polypeptides can be combined with another drug or agent (such as a protein, polypeptide, antibody, antibody fragment that may or may not be an anticancer drug or agent), such as an anticancer drug or agent such as a TKI, a platinum-based drug or a drug or agent that targets EGFR. In another aspect, the compositions are combined with a MARCKS protein, polypeptide or fragment thereof, wherein the MARCKS fragment comprises a polypeptide fragment that does not overlap in amino acid sequence with a polypeptide of the present disclosure or the MPS polypeptides disclosed in International PCT Publication Nos. WO 2015/013669 and WO 2015/095789. These compositions can be combined with a carrier, such as a pharmaceutically acceptable carrier for use in the diagnostic, screening and therapeutic methods as disclosed herein.

This disclosure also provides pharmaceutical composition for in vitro and in vivo use comprising, or alternatively consisting essentially of, or yet further consisting of a therapeutically effective amount of the MPS polypeptide or polynucleotide encoding the MPS polypeptide, that causes at least about 75%, or alternatively at least about 80%, or alternatively at least about 85%, or alternatively at least about 90%, or alternatively at least about 95%, or alternatively at least about 99% effectiveness in the methods provided herein when applied in a molar concentration of less than about 10 micromolar, or alternatively less than about 9 micromolar, or alternatively less than about 8 micromolar, or alternatively less than about 7 micromolar, or alternatively less than about 6 micromolar, or alternatively less than about 5 micromolar, or alternatively less than about 4 micromolar, or alternatively less than about 3 micromolar, or alternatively less than about 2 micromolar, or alternatively less than about 1 micromolar, or alternatively less than about 0.5 micromolar, or alternatively less than about 0.25 micromolar concentration, as compared to a control that does not receive the composition. Comparative effectiveness can be determined by suitable in vitro or in vivo methods as known in the art.

This disclosure also provides compositions for in vitro and in vivo use comprising, or alternatively consisting essentially of, or yet further consisting of one or more of the isolated polypeptides or polynucleotides described herein and a pharmaceutically acceptable carrier. In one aspect, the compositions are pharmaceutical formulations for use in the therapeutic methods of this disclosure. In a further aspect, the disclosure provides a pharmaceutical composition comprising, or alternatively consisting essentially of, or yet further consisting of, the isolated polypeptide or polynucleotide in a concentration such that a therapeutically effective amount of the polypeptide or a pharmacological dose of the composition causes at least a 75%, or alternatively at least a 80%, or alternatively at least a 85%, or alternatively at least a 90%, or alternatively at least a 95% or alternatively at least a 97% reduction in cell growth for example, when applied in a molar concentration of less than 1 micromolar, to a culture of responsive cancer cells as compared to a control that does not receive the composition.

### Isolated Polynucleotides and Compositions

This disclosure also provides isolated polynucleotides encoding the polypeptides described above. In one aspect, this disclosure also provides isolated polynucleotides encoding the polypeptides described above and an isolated anti-MPS shRNA. Non-limiting examples of the polypeptides of this disclosure include SEQ ID Nos. 40-56, 58 and 59 and equivalents thereof. This disclosure also provides the complementary polynucleotides to the sequences identified above, and their equivalents. Complementarity can be determined using traditional hybridization under conditions of moderate or high stringency. In one aspect the polynucleotides encode the equivalents of the isolated polypeptides of this disclosure. In another aspect, provided herein are equivalents of the isolated polynucleotides or their complements, wherein the equivalents have at least 80% sequence identity to the polynucleotides of this disclosure.

An equivalent of the isolated polynucleotide or its complement comprises or alternatively consists essentially of, or yet consists of a polynucleotide having at least 80% sequence identity to a polynucleotide encoding the isolated polypeptides of this disclosure or their equivalents that hybridizes to an isolated polynucleotide that encodes the isolated polypeptide or its complement. Also provided are polynucleotides encoding substantially homologous and equivalent polypeptides or peptide fragments. Substantially homologous and equivalent intends those having varying degrees of homology, such as at least 65%, or alternatively, at least 70%, or alternatively, at least 75%, or alternatively at least 80%, or alternatively, at least 85%, or alternatively at least 90%, or alternatively, at least 95%, or alternatively at least 97% homologous as defined above and which encode polypeptides having the biological activity as described herein. It should be understood although not always explicitly stated that embodiments to substantially homologous peptides and polynucleotides are intended for each aspect of this disclosure, e.g., peptides, polynucleotides and antibodies.

Alternatively, an equivalent is a polypeptide encoded by a nucleic acid that hybridizes under stringent conditions to a nucleic acid or complement that encodes the polypeptide or when a polynucleotide, a polynucleotide that hybridizes to the reference polynucleotide or its complement under conditions of high stringency. Equivalent polynucleotides hybridize under conditions of high stringency to a polynucleotide encoding the polypeptide of this disclosure or its equivalent, or the complement of each. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell. An equivalent polynucleotide is one that hybridizes under stringent conditions to the reference polynucleotide or the complement of the reference polynucleotide, an in one aspect, having similar biological activity as the reference polynucleotide.

In a further aspect, the polynucleotides and their complements and the equivalents of each thereof are labeled with a detectable marker or label, such as a dye or radioisotope, for ease of detection. The polynucleotides can be inserted into expression vectors and delivered into target cells, e.g., cancer cells, for the diagnostic and therapeutic methods as disclosed herein.

As used herein, the term polynucleotide intends DNA and RNA as well as modified nucleotides. For example, this disclosure also provides the anti-sense polynucleotide strand, e.g. antisense RNA or siRNA (shRNA) to these sequences or their complements. One can obtain an antisense RNA using the sequences that encode MPS polypeptide a methodology known to one of ordinary skill in the art wherein the degeneracy of the genetic code provides several polynucleotide sequences that encode the same polypeptide or the methodology described in Van der Krol et al. (1988) BioTechniques 6:958.

The polynucleotides of this disclosure can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this disclosure also provides a process for obtaining the peptide fragments of this disclosure by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

In one aspect, the polynucleotide is an RNA molecule that is short interfering RNA, also known as siRNA. Methods to prepare and screen interfering RNA and select for the ability to block polynucleotide expression are known in the art and non-limiting examples of which are shown below. These interfering RNA are provided by this disclosure alone or in combination with a suitable vector or within a host cell. Compositions containing the RNAi are further provided. RNAi is useful to knock-out or knock-down select functions in a cell or tissue as known in the art and described herein.

siRNA sequences can be designed by obtaining the target mRNA sequence and determining an appropriate siRNA complementary sequence. siRNAs of the disclosure are designed to interact with a target sequence, meaning they complement a target sequence sufficiently to hybridize to that sequence. An siRNA can be 100% identical to the target sequence. However, homology of the siRNA sequence to the target sequence can be less than 100% as long as the siRNA can hybridize to the target sequence. Thus, for example, the siRNA molecule can be at least 70%, 75%, 80%, 85%, 90%, 95%, 96%, 97%, 98%, 99% or 100% identical to the target sequence or the complement of the target sequence. Therefore, siRNA molecules with insertions, deletions or single point mutations relative to a target may also be used. The generation of several different siRNA sequences per target mRNA is recommended to allow screening for the optimal target sequence. A homology search, such as a BLAST search, should be performed to ensure that the siRNA sequence does not contain homology to any known mammalian gene.

In general, it is preferable that the target sequence be located at least 100-200 nucleotides from the AUG initiation codon and at least 50-100 nucleotides away from the termination codon of the target mRNA (Duxbury (2004) J. Surgical Res. 117:339-344).

Researchers have determined that certain characteristics are common in siRNA molecules that effectively silence their target gene (Duxbury (2004) J. Surgical Res. 117:339-344; Ui-Tei et al. (2004) Nucl. Acids Res. 32:936-48). As a general guide, siRNAs that include one or more of the following conditions are particularly useful in gene silencing in mammalian cells: GC ratio of between 45-55%, no runs of more than 9 G/C residues, G/C at the 5′ end of the sense strand; A/U at the 5′ end of the antisense strand; and at least 5 A/U residues in the first 7 bases of the 5′ terminal of the antisense strand.

siRNA are, in general, from about 10 to about 30 nucleotides in length. For example, the siRNA can be 10-30 nucleotides long, 12-28 nucleotides long, 15-25 nucleotides long, 19-23 nucleotides long, or 21-23 nucleotides long. When a siRNA contains two strands of different lengths, the longer of the strands designates the length of the siRNA. In this situation, the unpaired nucleotides of the longer strand would form an overhang.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. For example, the stem can be 10-30 nucleotides long, 12-28 nucleotides long, 15-25 nucleotides long, 19-23 nucleotides long, or 21-23 nucleotides long.

Tools to assist siRNA design are readily available to the public. For example, a computer-based siRNA design tool is available on the internet at www.dharmacon.com, last accessed on Nov. 26, 2007.

This disclosure also provides compositions for in vitro and in vivo use comprising, or alternatively consisting essentially of, or yet further consisting of one or more of the isolated polynucleotide as described herein and a pharmaceutically acceptable carrier. In one aspect, the compositions are pharmaceutical formulations for use in the therapeutic methods of this disclosure. In a further aspect, the disclosure provides a pharmaceutical composition comprising, or alternatively consisting essentially of, or yet further consisting of, the isolated polynucleotide in a concentration such that a therapeutically effective amount of the or pharmacological dose of the composition causes at least a 75%, or alternatively at least a 80%, or alternatively at least a 85%, or alternatively at least a 90%, or alternatively at least a 95% or alternatively at least a 97% reduction in cancer cell growth, viability or migration, as compared to a control that does not receive the composition. Comparative effectiveness can be determined by suitable in vitro or in vivo methods as known in the art and described herein.

### Synthesis of dsRNA and siRNA

dsRNA and siRNA can be synthesized chemically or enzymatically in vitro as described in Micura (2002) Agnes Chem. Int. Ed. Emgl. 41:2265-2269; Betz (2003) Promega Notes 85:15-18; and Paddison and Hannon (2002) Cancer Cell. 2:17-23. Chemical synthesis can be performed via manual or automated methods, both of which are well known in the art as described in Micura (2002), supra. siRNA can also be endogenously expressed inside the cells in the form of shRNAs as described in Yu et al. (2002) Proc. Natl. Acad. Sci. USA 99:6047-6052; and McManus et al. (2002) RNA 8:842-850. Endogenous expression has been achieved using plasmid-based expression systems using small nuclear RNA promoters, such as RNA polymerase III U6 or H1, or RNA polymerase II U1 as described in Brummelkamp et al. (2002) Science 296:550-553 (2002); and Novarino et al. (2004) J. Neurosci. 24:5322-5330.

In vitro enzymatic dsRNA and siRNA synthesis can be performed using an RNA polymerase mediated process to produce individual sense and antisense strands that are annealed in vitro prior to delivery into the cells of choice as described in Fire et al. (1998) Nature 391:806-811; Donze and Picard (2002) Nucl. Acids Res. 30(10): e46; Yu et al. (2002); and Shim et al. (2002) J. Biol. Chem. 277:30413-30416. Several manufacturers (Promega, Ambion, New England Biolabs, and Stragene) produce transcription kits useful in performing the in vitro synthesis.

In vitro synthesis of siRNA can be achieved, for example, by using a pair of short, duplex oligonucleotides that contain T7 RNA polymerase promoters upstream of the sense and antisense RNA sequences as the DNA template. Each oligonucleotide of the duplex is a separate template for the synthesis of one strand of the siRNA. The separate short RNA strands that are synthesized are then annealed to form siRNA as described in Protocols and Applications, Chapter 2: RNA interference, Promega Corporation, (2005).

In vitro synthesis of dsRNA can be achieved, for example, by using a T7 RNA polymerase promoter at the 5′-ends of both DNA target sequence strands. This is accomplished by using separate DNA templates, each containing the target sequence in a different orientation relative to the T7 promoter, transcribed in two separate reactions. The resulting transcripts are mixed and annealed post-transcriptionally. DNA templates used in this reaction can be created by PCR or by using two linearized plasmid templates, each containing the T7 polymerase promoter at a different end of the target sequence. Protocols and Applications, Chapter 2: RNA interference, Promega Corporation (2005).

RNA can be obtained by first inserting a DNA polynucleotide into a suitable prokaryotic or eukaryotic host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook and Russell (2001) supra. For instance, mRNA can be isolated using various lytic enzymes or chemical solutions according to the procedures set forth in Sambrook and Russell (2001) supra or extracted by nucleic-acid-binding resins following the accompanying instructions provided by manufactures.

In order to express the proteins described herein, delivery of nucleic acid sequences encoding the gene of interest can be delivered by several techniques. Examples of which include viral technologies (e.g. retroviral vectors, adenovirus vectors, adeno-associated virus vectors, alphavirus vectors and the like) and non-viral technologies (e.g. DNA/liposome complexes, micelles and targeted viral protein-DNA complexes) as described herein. Once inside the cell of interest, expression of the transgene can be under the control of ubiquitous promoters (e.g. EF-1) or tissue specific promoters (e.g. Calcium Calmodulin kinase 2 (CaMKI) promoter, NSE promoter and human Thy-1 promoter). Alternatively, expression levels may be controlled by use of an inducible promoter system (e.g. Tet on/off promoter) as described in Wiznerowicz et al. (2005) Stem Cells 77:8957-8961.

Non-limiting examples of promoters include, but are not limited to, the cytomegalovirus (CMV) promoter (Kaplitt et al. (1994) Nat. Genet. 8:148-154), CMV/human ÿ-globin promoter (Mandel et al. (1998) J. Neurosci. 18:4271-4284), NCX1 promoter, ÿMHC promoter, MLC2v promoter, GFAP promoter (Xu et al. (2001) Gene Ther. 8:1323-1332), the 1.8-kb neuron-specific enolase (NSE) promoter (Klein et al. (1998) Exp. Neurol. 150:183-194), chicken beta actin (CBA) promoter (Miyazaki (1989) Gene 79:269-277) and the β-glucuronidase (GUSB) promoter (Shipley et al. (1991) Genetics 10:1009-1018), the human serum albumin promoter, the alpha-1-antitrypsin promoter. To improve expression, other regulatory elements may additionally be operably linked to the transgene, such as, e.g., the Woodchuck Hepatitis Virus Post-Regulatory Element (WPRE) (Donello et al. (1998) J. Virol. 72: 5085-5092) or the bovine growth hormone (BGH) polyadenylation site.

The disclosure further provides the isolated polynucleotides of this disclosure operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells as described above and constructed using well known methods. See Sambrook and Russell (2001), supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; DEAE-dextran; electroporation; or microinjection. See Sambrook and Russell (2001), supra for this methodology.

The present disclosure also provides delivery vehicles suitable for delivery of a polynucleotide of the disclosure into cells (whether in vivo, ex vivo, or in vitro). A polynucleotide of the disclosure can be contained within a gene delivery vehicle, a cloning vector or an expression vector. These vectors (especially expression vectors) can in turn be manipulated to assume any of a number of forms which may, for example, facilitate delivery to and/or entry into a cell.

In one aspect when polynucleotides encoding two or more peptides, at least one of which is an MPS, SEQ ID NO: 40-56, 58 and 59, or an equivalent of each thereof, are intended to be translated and optionally expressed, the polynucleotides encoding the polypeptides may be organized within a recombinant mRNA or cDNA molecule that results in the transcript that expresses on a single mRNA molecule the at least two peptides. This is accomplished by use of a polynucleotide that has the biological activity of an internal ribosome entry site (IRES) located between the polynucleotide encoding the two peptides. IRES elements initiate translation of polynucleotides without the use of a “cap” structure traditionally thought to be necessary for translation of proteins in eukaryotic cells. Initially described in connection with the untranslated regions of individual picornaviruses, e.g. polio virus and encephalomyocarditis virus, IRES elements were later shown to efficiently initiate translation of reading frames in eukaryotic cells and when positioned downstream from a eukaryotic promoter, it will not influence the “cap”-dependent translation of the first cistron. The IRES element typically is at least 450 nucleotides long when in occurs in viruses and possesses, at its 3′ end, a conserved “UUUC” sequence followed by a polypyrimidine trace, a G-poor spacer and an AUG triple.

As used herein, the term “IRES” is intended to include any molecule such as a mRNA polynucleotide or its reverse transcript (cDNA) which is able to initiate translation of the gene downstream from the polynucleotide without the benefit of a cap site in a eukaryotic cell. “IRES” elements can be identical to sequences found in nature, such as the picornavirus IRES, or they can be non-naturally or non-native sequences that perform the same function when transfected into a suitable host cell. Bi- and poly-cistronic expression vectors containing naturally occurring IRES elements are known in the art and described for example, in Pestova et al. (1998) Genes Dev. 12:67-83 and International PCT Publication No. WO 01/04306, which in turn on page 17, lines 35 to 38 references several literature references which include, but are not limited to Ramesh et al. (1996) Nucl. Acids Res. 24: 2697-2700; Pelletier et al. (1988) Nature 334:320-325; Jan et al. (1989) J. Virol. 63:1651-1660; and Davies et al. (1992) J. Virol. 66:1924-1932. Paragraph [0009] of U.S. Patent Application Publication No. 2005/0014150 A1 discloses several issued U.S. patents wherein a virally-derived IRES element was used to express foreign gene(s) in linear multi-cistronic mRNAs in mammalian cells, plant cells and generally in eukaryotic cells. U.S. Patent Application Publication No. 2004/0082034 A1 discloses an IRES element active in insect cells. Methods to identify new elements also are described in U.S. Pat. No. 6,833,254.

Also within the term “IRES” element are cellular sequences similar to that disclosed in U.S. Pat. No. 6,653,132. The patent discloses a sequence element (designated SP163) composed of sequences derived from the 5′-UTR of VEGF (Vascular Endothelial Growth Factor gene), which, was presumably generated through a previously unknown mode of alternative splicing. The patentees report that an advantages of SP163 is that it is a natural cellular IRES element with a superior performance as a translation stimulator and as a mediator of cap-independent translation relative to known cellular IRES elements and that these functions are maintained under stress conditions.

Further within the term “IRES” element are artificial sequences that function as IRES elements that are described, for example, in U.S. Patent Application Publication No. 2005/0059004 A1.

Operatively linked to the IRES element and separately, are sequences necessary for the translation and proper processing of the peptides. Examples of such include, but are not limited to a eukaryotic promoter, an enhancer, a termination sequence and a polyadenylation sequence. Construction and use of such sequences are known in the art and are combined with IRES elements and protein sequences using recombinant methods. “Operatively linked” shall mean the juxtaposition of two or more components in a manner that allows them to junction for their intended purpose. Promoters are sequences which drive transcription of the marker or target protein. It must be selected for use in the particular host cell, i.e., mammalian, insect or plant. Viral or mammalian promoters will function in mammalian cells. The promoters can be constitutive or inducible, examples of which are known and described in the art.

In one aspect, the peptides are transcribed and translated from a separate recombinant polynucleotide and combined into a functional protein in the host cell. This recombinant polynucleotide does not require the IRES element or marker protein although in one aspect, it may be present.

These isolated host cells containing the polynucleotides of this disclosure are useful in the methods described herein as well as for the recombinant replication of the polynucleotides and for the recombinant production of peptides and for high throughput screening.

### Vectors and Host Cells

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a gene of interest. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

Provided herein is a vector comprising, or alternatively consisting essentially of, or yet further consisting of one or more of the isolated polynucleotide of this disclosure and optionally regulatory sequences operatively linked to the isolated polynucleotide for replication and/or expression. Non-limiting examples of a vector include a plasmid or a viral vector such as a retroviral vector, a lentiviral vector, an adenoviral vector, or an adeno-associated viral vector. In one particular aspect, the vector is an AAV vector (adeno-associated viral vector).

In one aspect, the regulatory sequences comprise, or alternatively consist essentially of, or yet further consist of a promoter, an enhancer element and/or a reporter. In one aspect, the vector further comprises, or alternatively consists essentially of, or yet further consists of a detectable marker or a purification marker.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly (NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

Further disclosed herein is a host cell further comprising or alternatively consisting essentially of, or yet further consisting one or more of the isolated polypeptide, the isolated polynucleotide, or the vector of this disclosure.

Suitable cells containing the polypeptides and/or polynucleotides include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

In addition to species specificity, the cells can be of any particular tissue type such as a somatic or embryonic stem cell such as a stem cell that can or cannot differentiate into a terminally differentiated cell. The stem cell can be of human or animal origin, such as mammalian.

### Antibody Compositions

This disclosure also provides an antibody capable of specifically forming a complex with a polypeptide of this disclosure, e.g. a polypeptide of SEQ ID Nos: 40-56 which can be used for screening for said polypeptides. In one aspect, the antibody or fragment thereof specifically binds to a phosphorylation site domain (PSD) of MARCKS protein, which can prevent MARCKS from phosphorylation and/or sequester the proteins that naturally interact with MARCKS. In another aspect, the antibody or fragment thereof is conjugated to a peptide or other molecule to facilitate entry into the cell. The term “antibody” is described above and includes polyclonal antibodies and monoclonal antibodies, antibody fragments, as well as derivatives thereof. The antibodies include, but are not limited to cows, rabbits, goats, mice, rats, hamsters, guinea pigs, sheep, dogs, cats, monkeys, chimpanzees, apes, etc. The antibodies are also useful to identify and purify therapeutic and/or diagnostic polypeptides. Also provided are hybridoma cell lines producing monoclonal antibodies of this disclosure.

Polyclonal antibodies of the disclosure can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammal's serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the disclosure can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134.

Antibody derivatives of the present disclosure can also be prepared by delivering a polynucleotide encoding an antibody of this disclosure to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this disclosure to provide transgenic plants and cultured plant cells (e.g., but not limited to tobacco, maize, and duckweed) that produce such antibodies, specified portions or variants in the plant parts or in cells cultured there from. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize has been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies of the present disclosure can also be produced using transgenic plants, according to known methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present disclosure can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See, e.g., Russel et al. (2000) Infection and Immunity April 2000:1820-1826; Gallo et al. (2000) European J. of Immun. 30:534-540; Green (1999) J. of Immun. Methods 231:11-23; Yang et al. (1999) J. of Leukocyte Biology 66:401-410; Yang (1999) Cancer Research 59(6):1236-1243; Jakobovits (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this disclosure also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this disclosure can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in other mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}—C_{H} 1-VH-C_{H}1) which form a pair of antigen binding regions. Linear antibodies can be bispecific or monospecific.

The antibodies of this disclosure can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from prokaryotic cells as described above.

If a monoclonal antibody being tested binds with protein or polypeptide, then the antibody being tested and the antibodies provided by the hybridomas of this disclosure are equivalents. It also is possible to determine without undue experimentation, whether an antibody has the same specificity as the monoclonal antibody of this disclosure by determining whether the antibody being tested prevents a monoclonal antibody of this disclosure from binding the protein or polypeptide with which the monoclonal antibody is normally reactive. If the antibody being tested competes with the monoclonal antibody of the disclosure as shown by a decrease in binding by the monoclonal antibody of this disclosure, then it is likely that the two antibodies bind to the same or a closely related epitope. Alternatively, one can pre-incubate the monoclonal antibody of this disclosure with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this disclosure.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307.

The isolation of other hybridomas secreting monoclonal antibodies with the specificity of the monoclonal antibodies of the disclosure can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

Antibodies can be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

The antibodies of the disclosure also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding monoclonal antibodies, or will be able to ascertain such, using routine experimentation.

### Compositions for Therapy

Provided herein is a composition comprising, or alternatively consisting essentially of, or yet further consisting of a carrier and one or more of the isolated polypeptide, the isolated polynucleotide, the vector or the host cell of this disclosure, e.g., in one aspect, the composition comprises as isolated polypeptide of SEQ ID Nos: 1-59, or alternatively SEQ ID Nos: 40-56, 59, or 40-56, 58 and 59, or a polynucleotide that encodes the polypeptide, or an equivalent of each thereof. Further diagnostic compositions include and antibody that binds the polypeptide or its equivalent or a fragment thereof. In one aspect, the carrier is a pharmaceutically acceptable carrier. In a further aspect, one or more of the above antibody, antibody fragment, antibody derivative, polypeptide or polynucleotides encoding these compositions and siRNA, vector, or host cell can be further comprise, or alternatively consist essentially of, or yet further consist of a chemotherapeutic agent or drug, or an anti-fibrotic agent or drug. Non-limiting examples of anti-fibrotic agent or drug include pirfenidone and nintedanib. Non-limiting examples of chemotherapeutic agent or drug include a Tyrosine Kinase Inhibitor (TKI), a platinum-based drug, a drug or agent that targets EGFR, or a MANS polypeptide or fragment thereof, wherein the fragment comprises, or alternatively consists essentially of, or yet further consists of a polypeptide and a carrier, a pharmaceutically acceptable carrier or medical device which is suitable for use of the compositions in diagnostic or therapeutic methods. Thus, the compositions comprise, or alternatively consist essentially of, or yet further consist of, one or more of the above compositions described above in combination with a carrier, a pharmaceutically acceptable carrier or medical device.

The carrier can be a liquid phase carrier or a solid phase carrier, e.g., bead, gel, microarray, or carrier molecule such as a liposome. The composition can optionally further comprise at least one further compound, protein or composition.

Additional examples of “carriers” includes therapeutically active agents such as another peptide or protein (e.g., a Fab′ fragment). For example, an antibody of this disclosure, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this disclosure encompasses a large variety of antibody conjugates, bi- and multispecific molecules, and fusion proteins, whether or not they target the same epitope as the antibodies of this disclosure.

Additional examples of “carriers” also include therapeutically active agents such as another peptide or protein (e.g., an Fab′ fragment) or agent for the treatment of one or more of: suppressing MARCKS phosphorylation and/or dissociation from the cell membrane; suppressing or reducing Th2 cytokine (IL-4, IL-5, IL-13 and eotaxin) production and/or IgE level; suppressing mucous metaplasia; inhibiting or suppressing infiltration of inflammatory cells (monocytes, neutrophils, lymphocytes); a disease or disease symptoms associated with allergic inflammation or hyper-reactivity.

Yet additional examples of carriers are organic molecules (also termed modifying agents) or activating agents, that can be covalently attached, directly or indirectly, to a polypeptide, antibody, antibody fragment, antibody derivative, polynucleotide encoding these, or RNAi, vector or host cell of this disclosure. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the disclosure has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the disclosure can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-A9-octadecanoate, all cis-A5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The present disclosure provides a composition comprising, or alternatively consisting essentially of, or yet further consisting of, at least one antibody of this disclosure, derivative or fragment thereof, suitable for administration in an effective amount to inhibit the expression of MARCKS for preventing, reducing, delaying, inhibiting or suppressing disease or disease symptoms associated with MARCKS phosphorylation and/or dissociation from the cell membrane and/or PIP2-sequestering effect, or PIP3 production, or activation of AKT, or inflammation, fibrosis, or activated fibroblast proliferation, or myofibroblast genesis and differentiation, or transforming growth factor-beta (TGF-β) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic. In one aspect, the compositions have the ability to prevent, reduce, delay, inhibit or suppress disease or disease symptoms associated with lung fibrosis, idiopathic pulmonary fibrosis, or smoking, bleomycin-induced pulmonary fibrosis, kidney fibrosis, liver fibrosis, skin fibrosis, fibroblastic lesions, activated fibroblast proliferation, inflammation, or myofibroblast genesis. In another aspect, the compositions have the ability to prevent, reduce, delay, inhibit or suppress disease or disease symptoms associated with lymphoma, leukemia or a solid tumor. Non-limiting examples of solid tumor include cancer, kidney cancer, brain cancer, colorectal cancer, pancreatic cancer, bone cancer, or throat cancer.

The compositions include, for example, pharmaceutical and diagnostic compositions/kits, comprising a pharmaceutically acceptable carrier and at least one antibody of this disclosure, variant, derivative or fragment thereof. As noted above, the composition can further comprise additional antibodies or therapeutic agents which in combination, provide multiple therapies tailored to provide the maximum therapeutic benefit.

Alternatively, a composition of this disclosure can be co-administered with other therapeutic agents, such as a small molecule or peptide, whether or not linked to them or administered in the same dosing. They can be co-administered simultaneously with such agents (e.g., in a single composition or separately) or can be administered before or after administration of such agents.

### Compositions for Diagnosis

One or more of the above compositions can be further combined with a carrier, a pharmaceutically acceptable carrier or medical device which is suitable for use of the compositions in diagnostic or therapeutic methods. In one aspect, the composition comprises as isolated polypeptide of SEQ ID Nos: 1-59, or alternatively SEQ ID Nos: 40-59, or alternatively SEQ ID Nos: 40-56, 58 and 59, or a polynucleotide that encodes the polypeptide, or an equivalent of each thereof. Further diagnostic compositions include and antibody that binds the polypeptide or its equivalent or a fragment thereof.

The carrier can be a liquid phase carrier or a solid phase carrier, e.g., bead, gel, gene chip, microarray, or carrier molecule such as a liposome. The composition can optionally further comprise, or alternatively consist essentially of, or yet further consist of at least one further compound, protein or composition, anticancer agent or other small molecule, protein, polypeptide, antibody or antibody fragment, e.g., a TKI inhibitor, a drug or agent that targets EGFR, a platinum-based drug or a MARCKS polypeptide or fragment thereof.

Additional examples of “carriers” includes therapeutically active agents such as another peptide or protein (e.g., a Fab′ fragment). For example, an antibody, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Additionally, the antibodies or fragments thereof can be linked to the polypeptides of this disclosure to facilitate targeting to a cell or tissue of choice and/or to stabilize the polypeptide. Accordingly, this disclosure encompasses a large variety of antibody conjugates, bi- and multispecific molecules, and fusion proteins, whether or not they target the same epitope as the antibodies of this disclosure.

Yet additional examples of carriers are organic molecules (also termed modifying agents) or activating agents, that can be covalently attached, directly or indirectly, to an antibody of this disclosure. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the disclosure has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the disclosure can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-A9-octadecanoate, all cis-A5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

Also provided is a composition containing at least one antibody of this disclosure. The compositions include, for example, pharmaceutical and diagnostic compositions/kits, comprising a pharmaceutically acceptable carrier and at least one antibody of this disclosure, variant, derivative or fragment thereof. As noted above, the composition can further comprise additional antibodies or therapeutic agents which in combination, provide multiple therapies tailored to provide the maximum therapeutic benefit.

Alternatively, a composition of this disclosure can be co-administered with other therapeutic agents, whether or not linked to them or administered in the same dosing. They can be co-administered simultaneously with such agents (e.g., in a single composition or separately) or can be administered before or after administration of such agents. Such agents can include anticancer therapies such as erlotinib, irinotecan, 5-Fluorouracil, Erbitux, Cetuximab, FOLFOX, or radiation therapy or other agents known to those skilled in the art.

### Diagnostic Methods Utilizing Recombinant DNA Technology and Bioinformatics

The polynucleotides of this disclosure can be attached to a solid support such as an array or high density chip for use in high throughput screening assays using methods known in the art. For example, a polynucleotide encoding MPS, e.g. SEQ ID NOs: 1-59, or alternatively 40-56, or alternatively SEQ ID Nos: 40-56, 58 and 59, or an equivalent of each thereof can be used as a probe to identify expression in a subject sample. The chips can be synthesized on a derivatized glass surface using the methods disclosed in U.S. Pat. Nos. 5,405,783; 5,412,087 and 5,445,934. Photoprotected nucleoside phosphoramidites can be coupled to the glass surface, selectively deprotected by photolysis through a photolithographic mask, and reacted with a second protected nucleoside phosphoramidite. The coupling/deprotection process is repeated until the desired probe is complete.

One can use chemical synthesis to provide the isolated polynucleotides of the present disclosure. Chemical synthesis of polynucleotides can be accomplished using a number of protocols, including the use of solid support chemistry, where an oligonucleotide is synthesized one nucleoside at a time while anchored to an inorganic polymer. The first nucleotide is attached to an inorganic polymer using a reactive group on the polymer which reacts with a reactive group on the nucleoside to form a covalent linkage. Each subsequent nucleoside is then added to the first nucleoside molecule by: 1) formation of a phosphite linkage between the original nucleoside and a new nucleoside with a protecting group; 2) conversion of the phosphite linkage to a phosphate linkage by oxidation; and 3) removal of one of the protecting groups to form a new reactive site for the next nucleoside as described in U.S. Pat. Nos. 4,458,066; 5,153,319; 5,132,418; and 4,973,679, all of which are incorporated by reference herein. Solid phase synthesis of oligonucleotides eliminates the need to isolate and purify the intermediate products after the addition of every nucleotide base. Following the synthesis of RNA, the oligonucleotides is deprotected (U.S. Pat. No. 5,831,071) and purified to remove by-products, incomplete synthesis products, and the like.

U.S. Pat. No. 5,686,599, describes a method for one pot deprotection of RNA under conditions suitable for the removal of the protecting group from the 2′ hydroxyl position. U.S. Pat. No. 5,804,683, describes a method for the removal of exocyclic protecting groups using alkylamines. U.S. Pat. No. 5,831,071, describes a method for the deprotection of RNA using ethylamine, propylamine, or butylamine. U.S. Pat. No. 5,281,701, describes methods and reagents for the synthesis of RNA using 5′-O-protected-2′-O-alkylsilyl-adenosine phosphoramidite and 5′-O-protected-2′-O-alkylsilylguanosine phosphoramidite monomers which are deprotected using ethylthiotetrazole. Usman and Cedergren (1992) Trends in Biochem. Sci. 17:334-339 describe the synthesis of RNA-DNA chimeras for use in studies of the role of 2′ hydroxyl groups. Sproat et al. (1995) Nucleosides & Nucleotides 14:255-273, describe the use of 5-ethylthio-1H-tetrazole as an activator to enhance the quality of oligonucleotide synthesis and product yield. Gait et al. (1991) Oligonucleotides and Analogues, ed. F. Eckstein, Oxford University Press 25-48, describe general methods for the synthesis of RNA. U.S. Pat. Nos. 4,923,901; 5,723,599; 5,674,856; 5,141,813; 5,419,966; 4,458,066; 5,252,723; Weetall et al. (1974) Methods in Enzymology 34:59-72; Van Aerschot et al. (1988) Nucleosides and Nucleotides 7:75-90; Maskos and Southern (1992) Nucleic Acids Research 20: 1679-1684; Van Ness et al. (1991) Nucleic Acids Research 19:3345-3350; Katzhendler et al. (1989) Tetrahedron 45:2777-2792; Hovinen et al. (1994) Tetrahedron 50:7203-7218; GB 2,169,605; EP 325,970; International PCT Publication No. WO 94/01446; German Patent No. 280,968; and BaGerman Patent No. 4,306,839, all describe specific examples of solid supports for oligonucleotide synthesis and specific methods of use for certain oligonucleotides. Additionally, methods and reagents for oligonucleotide synthesis as known to one of skill in the art as describe by U.S. Pat. No. 7,205,399, incorporated herein by reference in its entirety.

The probes and high density oligonucleotide probe arrays also provide an effective means of monitoring expression of a multiplicity of genes, one of which includes the gene. Thus, the expression monitoring methods can be used in a wide variety of circumstances including detection of disease, identification of differential gene expression between samples isolated from the same patient over a time course, or screening for compositions that upregulate or downregulate the expression of the gene at one time, or alternatively, over a period of time.

Detectable labels suitable for use in the present disclosure include those identified above as well as any composition detectable by spectroscopic, photochemical, biochemical, immunochemical, electrical, optical or chemical means. Useful labels in the present disclosure include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P) enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

International PCT Publication No. WO 97/10365 describes methods for adding the label to the target (sample) nucleic acid(s) prior to or alternatively, after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids, see Laboratory Techniques In Biochemistry And Molecular Biology, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using the methods disclosed in International PCT Publication No. WO 97/10365.

Results from the chip assay are typically analyzed using a computer software program. See, for example, EP 0717 113 A2 and WO 95/20681. This information is compared against existing data sets of gene expression levels for diseased and healthy individuals. A correlation between the obtained data and that of a set of diseased individuals indicates the onset of a disease in the subject patient.

### Methods to Identify Therapeutic Agents

The present disclosure also provides methods to identify leads and methods for treating the disease or disease symptoms associated with one or more of: preventing, reducing, delaying, inhibiting or suppressing disease or disease symptoms associated with MARCKS phosphorylation and/or dissociation from the cell membrane and/or PIP2-sequestering effect, or PIP3 production, or activation of AKT, or inflammation, fibrosis, or activated fibroblast proliferation, or myofibroblast genesis and differentiation, or transforming growth factor-beta (TGF-β) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic. In one aspect, the compositions have the ability to prevent, reduce, delay, inhibit or suppress disease or disease symptoms associated with lung fibrosis, idiopathic pulmonary fibrosis, or smoking, bleomycin-induced pulmonary fibrosis, kidney fibrosis, liver fibrosis, skin fibrosis, fibroblastic lesions, activated fibroblast proliferation, inflammation, or myofibroblast genesis. In another aspect, the compositions have the ability to prevent, reduce, delay, inhibit or suppress disease or disease symptoms associated with lymphoma, leukemia or a solid tumor. Non-limiting examples of solid tumor include cancer, kidney cancer, brain cancer, colorectal cancer, pancreatic cancer, bone cancer, or throat cancer.

The present disclosure also provides methods to identify leads and methods for treating fibrosis and/or cancer. In one aspect, the screen identifies lead compounds or biologics agents that mimic the polypeptides identified above and which are useful to treat these disorders or to treat or ameliorate the symptoms associated with the disorders. Test substances for screening can come from any source. They can be libraries of natural products, combinatorial chemical libraries, biological products made by recombinant libraries, etc. The source of the test substances is not critical to the disclosure. The present disclosure provides means for screening compounds and compositions which may previously have been overlooked in other screening schemes.

To practice the screen or assay in vitro, suitable cell cultures or tissue cultures are first provided. The cell can be a cultured cell or a genetically modified cell which differentially expresses the receptor and/or receptor complex. Alternatively, the cells can be from a tissue culture as described below. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, suitable cells may be cultured in microtiter plates and several agents may be assayed at the same time by noting genotypic changes, phenotypic changes and/or cell death.

When the agent is a composition other than a DNA or RNA nucleic acid molecule, the suitable conditions may be by directly added to the cell culture or added to culture medium for addition. As is apparent to those skilled in the art, an “effective” amount must be added which can be empirically determined.

The screen involves contacting the agent with a test cell expressing the complex and then assaying the cell its ability to provide a biological response similar to the polypeptides of this disclosure. In yet another aspect, the test cell or tissue sample is isolated from the subject to be treated and one or more potential agents are screened to determine the optimal therapeutic and/or course of treatment for that individual patient.

For the purposes of this disclosure, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the screen. The agents and methods also are intended to be combined with other therapies. They can be administered concurrently or sequentially.

### Methods of Treatment

Provided herein are methods of treating disease or disease symptoms associated with fibrosis in a subject in need thereof, comprising, or alternatively consisting essentially of, or yet further consisting of administering to the subject an effective amount of one or more of the isolated polypeptide or the isolated polynucleotide of as identified above (e.g., SEQ. ID Nos: 1-59, or alternatively 40-59, or alternatively 40-56, 58 and 59) as well as a peptide or composition of the peptides of SEQ ID Nos: 1-59 or alternatively 40-59, or alternatively 40-56, 58 and 59, as well as a polypeptide comprising at least 6 and no more than 51 amino acids, wherein the amino acid sequence comprises, or alternatively consists essentially of, or alternatively consisting of a polypeptide of at least 6 amino acids to no more than 51 or alternatively 35 amino acids, comprising, or alternatively consisting essentially of, or yet consisting of SEQ ID Nos: 1-59 or alternatively 40-59, or alternatively 40-56, 58 and 59.

In one aspect, the peptide that comprises, or alternatively consists essentially of, or yet further consists of a peptide identified in the below table (SEQ ID NOS 48-54, 40-42, 45 and 47, respectively, in order of appearance (Red residues are D-isoforms of amino acids):

| Peptide | | |
|---|---|---|
| ID | Sequence | IC50 |
| MPS-11022 | KKKKKRFSFKKSFKLSGFSFKANKK | 83 |
| MPS-11011 | KKKKKRFSFKASFKLSGFSFKKNKK | 29 |
| MPS-11010 | KKKKKRFSFAKSFKLSGFSFKKNKK | 73 |
| MPS-11006 | KKKKKAFSFKKSFKLSGFSFKKNKK | 79 |
| MPS-11003 | KKAKKRFSFKKSFKLSGFSFKKNKK | 85 |
| MPS-11001 | AKKKKRFSFKKSFKLSGFSFKKNKK | 85 |
| MPS-11200 | Ac-KKKKKRFSFKKSFKLSGFSFKKNKK- | 78 |
| | NH2 | |
| MPS-21010 | FSFGSFSLKKFSFRKKKNKK | 83 |
| MPS-21020 | KKKKFSFGSFSLKKFSFRKKKNKK | 64 |
| MPS-21026 | KKKKFAFGAFALKKFAFRKKKNKK | 49 |
| MPS-12042 | KKKKKRFAFKKAFKLAGFAFKKNKK | 6 |
| MPS-22026 | KKKKKFAFGAFALKKFAFRKKKNKK | 11 |

In one aspect, the polypeptide is at least 6 amino acids and no more than 51 amino acids, or alternatively at least 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids biological equivalents of each thereof. In one aspect, a biological equivalent is a polypeptide wherein one or more amino acids have been substituted with a conservative amino acid substitution(s). In one aspect, all serines are replaced by alanines (A-MPSs). In a further aspect, myristic acid is conjugated or joined to the N-terminal amino acid of the peptides, including biological equivalents thereof, e.g., wherein all serines are replaced by alanines.

In a further aspect, the polypeptide is selected from an isolated polypeptide of SEQ ID NO: 18, wherein an amino acid corresponding to position 6 has been replaced with an alanine, proline, or glycine; or SEQ ID NO: 19, wherein an amino acid corresponding to position 7 has been replaced with an alanine, proline, or glycine; or SEQ ID NO: 20, wherein an amino acid corresponding to position 8 has been replaced with an alanine, proline, or glycine.

In one aspect of each of the above embodiments, D-MPS (wherein all serines are substituted with aspartate) and myristoylated-wild-type MPS are specifically excluded from the group of polypeptides and methods as disclosed herein.

In one aspect for the treatment of fibrosis, the “MPS” intends a polypeptide of at least 6 amino acids and no more than 51 amino acids, comprising, or alternatively consisting essentially of, or yet consisting of, SEQ ID Nos: 1-59, or alternatively 40-56, 58 and 59, where in some embodiments, and biological equivalents, wherein X is absent or is a basic amino acid, and/or Y is absent or a hydrophobic amino acid. In one aspect, the basic amino acid comprises one or more lysine (K), histidine (H) or arginine (R). In one aspect, all X are lysine (K). In one aspect, Y is one or more hydrophobic amino acids, selected from Alanine (A), Isoleucine (I), Leucine (L), Valine (V), Phenylalanine (F), Tryptophan (W) or Tyrosine (Y). In one aspect, all serines are alanines. In another aspect, all X are lysine and all S are substituted with alanine. In a further aspect, all S are Aspartate (D). In a yet further aspect, all of the above noted polypeptides as disclosed herein further comprise, or alternatively consist essentially of, or yet further consist of, myristic acid conjugated or joined to the N-terminal amino acid. In one aspect, MPS peptide comprises, or consists essentially of, or yet further aspect, the amino acid sequence. In one aspect, all serines are replaced by alanines (A-MPSs). In a further aspect, myristic acid is conjugated or joined to the N-terminal amino acid of SEQ ID NOS.: 1-59, or 40-59, or alternatively 40-56, 58 and 59, including biological equivalents thereof, e.g., wherein all serines are replaced by alanines.

The polypeptide can be no more than 51 amino acids, comprising, or alternatively consisting essentially of, or yet consisting of, an isolated polypeptide comprising, or alternatively consisting essentially of, or yet further consisting of, no more than 51 amino acids, wherein the amino acid sequence comprises SEQ ID Nos: 1-59, or 40-59, or alternatively 40-56, 58 and 59, and biological equivalents of each thereof, and wherein in one aspect, one or more of the serines (S) are substituted with one or more neutral or positively charged amino acids, that may be the same or different, e.g., alanines (A), glycines (G), or prolines (P), or a biological equivalent of each thereof, wherein a biological equivalent of comprises a polypeptide that has at least 80% sequence identity to the above polypeptides or amino acid sequences, or wherein a biological equivalent comprises an isolated polypeptide encoded by an isolated polynucleotide that hybridizes under high stringency conditions to the compliment polynucleotide encoding these polypeptide(s) or the polynucleotide encoding these polypeptides, and wherein high stringency hybridization conditions is generally performed at about 60° C. in about 1×SSC. In one aspect, term also includes the polypeptides having the amino acid sequence XXXRYAYXXAYX (SEQ ID NO: 58), wherein X is any amino acid, or XXXXXRYAYXXAYXLAGYAYXXNXX (SEQ ID NO: 59), wherein X is any amino acid and Y is a hydrophobic amino acid residue, including for example tyrosine, and optionally a polynucleotide comprising any contiguous 12 amino acid fragment of these sequences, and biological equivalents thereof; and further optionally wherein one or more serine (S) is substituted with one or more neutral or positively charged amino acids, that may be the same or different, e.g., one or more serines are substituted with one or more alanines (A), glycines (G), or prolines (P), and wherein each X is the same or different and is a basic amino acid and wherein each Y is the same or different and is a hydrophobic amino acid. Non-limiting examples of MPS polypeptides include an isolated polypeptide comprising a biological equivalent of SEQ ID NOs: 1-59, or alternatively 40-59, or alternatively 40-56, 58 and 59, which comprises a polypeptide that has at least 80% sequence identity to SEQ ID NOs: 1-59, or alternatively 40-59, or alternatively 40-56, 58 and 59, and optionally wherein one or more serine (S) is substituted with one or more neutral or positively charged amino acids, that may be the same or different, e.g., one or more serines are substituted with one or more alanines (A), glycines (G), or prolines (P), and/or wherein a biological equivalent comprises an isolated polypeptide encoded by an isolated polynucleotide that hybridizes under high stringency conditions to the compliment polynucleotide encoding SEQ ID NOs: 1-59, or alternatively 40-59, or alternatively 40-56, 58 and 59, and optionally wherein one or more serine (S) is substituted with one or more neutral or positively charged amino acids, that may be the same or different, e.g., one or more serines are substituted with one or more alanines (A), glycines (G), or prolines (P), and/or the polynucleotide encoding SEQ ID NOs: 1-59, or alternatively 40-59, or alternatively 40-56, 58 and 59, and optionally wherein one or more serine (S) is substituted with one or more neutral or positively charged amino acids, that may be the same or different, e.g., one or more serines are substituted with one or more alanines (A), glycines (G), or prolines (P), and wherein high stringency hybridization conditions is generally performed at about 60° C. in about 1×SSC. In one aspect, the basic amino acid comprises one or more lysine (K), histidine (H) or arginine (R). In one aspect, all X are lysine (K). In one aspect, Y is one or more hydrophobic amino acids, selected from alanine (A), isoleucine (I), leucine (L), valine (V), phenylalanine (F), tryptophan (W) or tyrosine (Y). In one aspect, the polypeptides as described above are no more than 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids or alternatively, the polypeptides of SEQ ID NO: 21, 25, 31 or 32, 40-56, 58 or 59, and optionally wherein one or more serine (S) is substituted with one or more neutral or positively charged amino acids, that may be the same or different, e.g., one or more serines are substituted with one or more alanines (A), glycines (G), or prolines (P), and wherein biological equivalents of each thereof.

The MPS polypeptides and biological equivalents have the ability to achieve the same or similar results as noted above. In one aspect, the basic amino acid comprises one or more lysine (K), histidine (H) or arginine (R). In one aspect, all X are lysine (K). In one aspect, Y is one or more hydrophobic amino acids, selected from alanine (A), isoleucine (I), leucine (L), valine (V), phenylalanine (F), tryptophan (W) or tyrosine (Y). In one aspect, the polypeptide is no more than 45 amino acids, or alternatively 40 amino acids, or alternatively 35 amino acids, or alternatively 30 amino acids, or alternatively no more than 25 amino acids, or alternatively no more than 20 amino acids, or alternatively no more than 15 amino acids or alternatively.

In one aspect, the polypeptides of SEQ ID NOs: 45 and 47, as compared to SEQ ID NOs: 46 and 48, are MPS polypeptides wherein the 4 serine residues of wild-type MPS peptide are replaced by alanine residues, e.g., (KKKKKRFAFKKAFKLAGFAFKKNKK (SEQ ID NO: 45), that increases membrane affinity. The polypeptides of SEQ ID NO: 45-48 are highly positive charged and interact electrostatically with PIP2 on the phospholipid membrane.

In one aspect, the disease or symptoms associated with fibrosis is selected from the group of: lung fibrosis, idiopathic pulmonary fibrosis, bleomycin-induced pulmonary fibrosis, kidney fibrosis, liver fibrosis, skin fibrosis, fibroblastic lesions, activated fibroblast proliferation, inflammation, or myofibroblast genesis.

Also provided herein are methods for one or more of inhibiting cancer cell growth, treating cancer, inhibiting metastasis, inhibiting cancer stem cell growth, inhibiting tumor cell mobility, restoring sensitivity of a resistant cancer cell to a chemotherapeutic agent, in a subject in need thereof, comprising administering to the subject an effective amount of one or more of the isolated polypeptide or the isolated polynucleotide of this disclosure. In one aspect, the cancer cell or cancer is lymphoma, leukemia or a solid tumor. In another aspect, the cancer cell or cancer is lung cancer, liver cancer, kidney cancer, brain cancer, colorectal cancer, pancreatic cancer, bone cancer, or throat cancer.

The present disclosure also provides methods to identify leads and methods for treating the disease or disease symptoms associated with one or more of: preventing, reducing, delaying, inhibiting or suppressing disease or disease symptoms associated with MARCKS phosphorylation and/or dissociation from the cell membrane and/or PIP2-sequestering effect, or PIP3 production, or activation of AKT, or inflammation, fibrosis, or activated fibroblast proliferation, or myofibroblast genesis and differentiation, or transforming growth factor-beta (TGF-β) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic.

In one aspect, the compositions have the ability to prevent, reduce, delay, inhibit or suppress disease or disease symptoms associated with lung fibrosis, idiopathic pulmonary fibrosis, or smoking, bleomycin-induced pulmonary fibrosis, kidney fibrosis, liver fibrosis, skin fibrosis, fibroblastic lesions, activated fibroblast proliferation, inflammation, or myofibroblast genesis. In another aspect, the compositions have the ability to prevent, reduce, delay, inhibit or suppress disease or disease symptoms associated with lymphoma, leukemia or a solid tumor. Non-limiting examples of solid tumor include cancer, kidney cancer, brain cancer, colorectal cancer, pancreatic cancer, bone cancer, or throat cancer.

Thus, methods to achieve such in vitro or in vivo are provided by contacting or administering an effective amount of the polypeptide and/or other therapeutic composition of this disclosure (e.g., antibody or siRNA) to a subject in need of such treatment. Administration can be by any suitable method and effective amounts can be empirically determined by a treating physician or one of skill in the art when the contacting is in vitro.

In a further aspect, the methods of treatment further comprise, or alternatively consist essentially of, or yet further consist of administering an effective amount of an anti-fibrotic agent or drug. Non-limiting examples of anti-fibrotic agent or drug include pirfenidone and nintedanib. Additional agents include but are not limited to nintedanib, oral prednisone (or some other form of corticosteroid), Fluimucil (N-acetylcysteine), Cytoxan (cyclophosphamide), a, combination of prednisone, azathioprine, and N-acetylcysteine (NAC), colchicine, D-penicillamine, pirfenidone (5-methyl-1-phenyl-2-[1H]-pyridone), interferon-β1a, relaxin, lovastatin, beractant, N-acetylcysteine, keratinocyte growth factor, captopril, hepatocyte growth factor, Rhokinase inhibitor, thrombomodulin-like protein, bilirubin, PPARγ (peroxisome proliferator-activated receptor gamma) activator, imatinib, and interferon-γ. In one aspect, the fibrosis is pulmonary fibrosis and the additional agents include one or more of colchicine, D-penicillamine, pirfenidone (5-methyl-1-phenyl-2-[1H]-pyridone), interferon-β1a, relaxin, lovastatin, beractant, N-acetylcysteine, keratinocyte growth factor, captopril, hepatocyte growth factor, Rhokinase inhibitor, thrombomodulin-like protein, bilirubin, PPARγ (peroxisome proliferator-activated receptor gamma) activator, imatinib, and interferon-γ. Additional agents are known in the literature, e.g., JP A No. 8-268906, WO 00/57913, JP A No. 2002-371006, JP A No. 2003-119138, JP A No. 2005-513031, JP A No. 2005-531628, JP A No. 2006-502153, WO 2006/068232, and Ann Intern Med. 2001; 134(2): 136-51.

In some embodiments, the subjects with IPF are “unresponsive to conventional treatment,” i.e., unresponsive to conventional prior art treatments of IPF including corticosteroids, cyclophosphamide, and azathioprine.

In another aspect, the methods of treatment further comprise, or alternatively consist essentially of, or yet further consist of administering an effective amount of an anti-cancer drug or agent.

In one aspect, the methods of treatment further comprise, or alternatively consist essentially of, or yet further consist of administering an effective amount of an anti-cancer drug or agent. In a further aspect, the methods of treatment further comprise, or alternatively consist essentially of, or yet further consist of administering an effective amount of a tyrosine kinase inhibitor, a platinum drug or an immunotherapeutic. In a yet further aspect, an effective amount of an agent or drug (chemotherapeutic or other) can be combined and contacted or administered as appropriate. In one aspect the chemotherapeutic is a TKI, or a platinum-based drug, or an agent that targets EGFR or yet further a MARCKS polypeptide or fragment thereof, wherein the fragment is not an N-terminal fragment of MARCKS or a polypeptide that does not have an amino acid sequence having sequence identity to a polypeptide as described above.

Also provided is a method for restoring sensitivity of a chemoresistant cancer cell to a chemotherapeutic drug, the method comprising or alternatively consisting essentially of, or yet further consists of, contacting the cell or administering to a subject in need thereof, an effective amount of an isolated MPS polypeptide or an equivalent thereof or an anti-MARCKS siRNA, and optionally, wherein the chemotherapeutic drug or agent is selected from a TKI, a platinum-based drug, a drug or agent that targets EGFR, cisplatin, paclitaxel, erlotinib or dasatinib; and optionally wherein the chemoresistant cancer cell is a TKI resistant cell. siRNA- and shRNA-MARCKS inhibiting RNA are known in the art (see, e.g., WO 2015/013669) and sequences provided herein. The contacting is in vitro or in vivo and in one aspect, the cell is a mammalian solid tumor cell. In one aspect, the tumor cell comprises or expresses higher levels of phosphorylated MARCKS polypeptide as compared to a normal counterpart cell. Non-limiting examples of such cells include a lung cancer cell, a colon cancer cell, a breast cancer cell or a pancreatic cancer and alternatively or in addition, the patient suffering from advanced cancer (Stage II to IV). In a further aspect, the method further comprises contacting the cell or administering to the patient or subject an effective amount of a chemotherapeutic drug or agent, e.g., a TKI, or a platinum-based drug or agent that targets EGFR, e.g., cisplatin, paclitaxel, erlotinib or dasatinib.

Further provided herein is a method of increasing efficacy of one or more of anti-fibrotic or anti-cancer agents or drugs for one or more of preventing, reducing, delaying, inhibiting or suppressing disease or disease symptoms associated with: MARCKS phosphorylation and/or dissociation from the cell membrane and/or PIP2-sequestering effect, or PIP3 production, or activation of AKT, or inflammation, or fibrosis, or fibroblastic lesions, or activated fibroblast proliferation, or myofibroblast genesis and differentiation, or transforming growth factor-beta (TGF-0) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic in a subject in need thereof, comprising administering to the subject an effective amount of one or more anti-fibrotic or anti-cancer agents or drugs in combination with an effective amount of an isolated polypeptide or isolated polynucleotide or compositions of this disclosure.

In one aspect, disclosed herein is a method of increasing efficacy of one or more of pirfenidone or nintedanib, or bemcentinib, or erlotinib for one or more of preventing, reducing, delaying, inhibiting or suppressing disease or disease symptoms associated with: MARCKS phosphorylation and/or dissociation from the cell membrane and/or PIP2-sequestering effect, or PIP3 production, or activation of AKT, or inflammation, or fibrosis, or lung fibrosis, or smoking, or idiopathic pulmonary fibrosis, or bleomycin-induced pulmonary fibrosis, or fibroblastic lesions, or activated fibroblast proliferation, or myofibroblast genesis and differentiation, or transforming growth factor-beta (TGF-β) signaling pathway, or cancer, or solid tumor cell growth or metastasis, or cancer stem cell growth, or tumor cell mobility; and optionally for promoting apoptosis, or restoring sensitivity of a resistant cancer cell to a chemotherapeutic in a subject in need thereof, comprising administering to the subject an effective amount of one or more of nintedanib, or bemcentinib, or erlotinib in combination with an effective amount of an isolated polypeptide or isolated polynucleotide or compositions of this disclosure. Non-limiting examples of cancer include lung cancer, liver cancer, kidney cancer, brain cancer, colorectal cancer, pancreatic cancer, bone cancer, throat cancer, lymphoma and leukemia.

In therapeutic applications, a pharmaceutical composition containing one or more polypeptide or other therapeutic composition (e.g., antibody or siRNA) described herein is administered to a patient suspected of, or already suffering from cancer, wherein said composition is administered in an amount sufficient to cure, or at least partially arrest, the symptoms of the disease (biochemical, histological and/or behavioral), including its complication and intermediate pathological phenotypes in development of the disease. In one aspect, administration is by intraperitoneal injection or orally.

In one particular aspect, disclosed herein is a method for delivering a polypeptide of this disclosure across the blood brain barrier in a subject in need thereof comprising, or alternatively consisting essentially of, or yet further consisting of administering an effective amount of vector as disclosed above to the subject. In one aspect, the peptide is delivered in the absence of an agent that promotes transport across the blood brain barrier, e.g., mannitol.

In one aspect, for the methods of treatment disclosed herein the administration is local to a tissue being treated or systemic. In one specific aspect, the local administration comprises, or alternatively consists essentially of, or yet further consists of topical or by inhalation therapy. In another aspect, the systemic administration is from the group of intravenous, intracranial, inhalation therapy, intranasal, vaginal or rectal administration.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell, solid tumor or cancer being treated, and the subject being treated. Single or multiple administrations can be carried out with the dose level and pattern being selected by the treating physician. Suitable dosage formulations and methods of administering the agents can be found below. Additional dosing strategies are disclosed in U.S. Pat. No. 10,039,515.

The pharmaceutical compositions can be administered orally, intranasally, parenterally, injection, orally and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present disclosure, the pharmaceutical compositions can also contain other pharmaceutically active compounds or a plurality of compounds of the disclosure.

More particularly, an agent of the present disclosure also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient. Desirable blood levels of the agent may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present disclosure suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present disclosure may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of the agent through the skin or other affected areas.

Examples of such dermal penetration enhancers include dimethylsulfoxide and related analogues.

The oily phase of the emulsions of this disclosure may be constituted from known ingredients in a known manner. While this phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present disclosure include Tween 60, Span 80, cetostearyl alcohol, myristyl alcohol, glyceryl monostearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above, the formulations of this disclosure may include other agents conventional in the art having regard to the type of formulation in question, for example, those suitable for oral administration may include such further agents as sweeteners, thickeners and flavoring agents. It also is intended that the agents, compositions and methods of this disclosure be combined with other suitable compositions and therapies.

The methods of this disclosure are used to treat “a subject,” “a host,” “an individual,” and “a patient” such as for example animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

As used herein, “treating” or “treatment” of a disease in a subject refers to (1) preventing the symptoms or disease from occurring in a subject that is predisposed or does not yet display symptoms of the disease; (2) inhibiting the disease or arresting its development or relapse; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

When the disease is fibrosis, the following clinical end points are non-limiting examples of treatment: reduction in fibrotic tissue, reduction in inflammation, reduction in fibroblastic lesions, reduction in activated fibroblast proliferation, reduction in myofibroblast genesis, reduction in rate of decline of Forced Vital Capacity (FVC), wherein FVC is the total amount of air exhaled during the lung function test, absolute and relative increases from baseline in FVC, absolute increase from baseline in FVC (% Predicted), increase in progression-free survival time, decrease from baseline in St George's Respiratory Questionnaire (SGRQ) total score, wherein SGRQ is a health-related quality of life questionnaire divided into 3 components: symptoms, activity and impact and the total score (summed weights) can range from 0 to 100 with a lower score denoting a better health status, and relative decrease from baseline in high resolution computerized tomography (HRCT) quantitative lung fibrosis (QLF) score, wherein the QLF score ranges from 0 to 100% and greater values represent a greater amount of lung fibrosis and are considered a worse health status. Non-limiting examples clinical end points for fibrosis treatment and tests that can be performed to measure said clinical end points are described in the following clinical trials: NCT03733444 (https://clinicaltrials.gov/ct2/show/NCT03733444), NCT00287729 (clinicaltrials.gov/ct2/show/NCT00287729), NCT00287716 (clinicaltrials.gov/ct2/show/NCT00287716), NCT02503657 (https://clinicaltrials.gov/ct2/show/NCT02503657), NCT00047645 (clinicaltrials.gov/ct2/show/NCT00047645), NCT02802345 (clinicaltrials.gov/ct2/show/NCT02802345), NCT01979952 (clinicaltrials.gov/ct2/show/NCT01979952), NCT00650091 (clinicaltrials.gov/ct2/show/NCT00650091), NCT01335464 (clinicaltrials.gov/ct2/show/NCT01335464), NCT01335477 (clinicaltrials.gov/ct2/show/NCT01335477), NCT01366209 (clinicaltrials.gov/ct2/show/NCT01366209). Further non-limiting examples clinical end points for fibrosis treatment and tests that can be performed to measure said clinical end points are described in King et al, N Engl J Med. (2014) May 29; 370(22):2083-92 and Richeldi et al, N Engl J Med. 2014 May 29; 370(22):2071-82.

### Kits

Also disclosed herein is a kit comprising, or alternatively consisting essentially of, or yet further consisting of one or more of: the isolated polypeptide, the isolated polynucleotide, the vector, or the composition of this disclosure and instructions for use. In one aspect, the instructions recite the methods of using the isolated polypeptide, the isolated polynucleotide, the vector, or the composition disclosed herein.

### EXPERIMENTAL

### Experiment No. 1

Lung fibrosis is an important step of normal lung injury-repair process since the lung is a primary target organ that is constantly bombarded with environmental air pollutants. Smoking is one of the etiologies in inducing lung injury and repair and with continuous smoking, causing uncontrolled lung injury and repair; this may lead to a life-threatening disease, such as idiopathic pulmonary fibrosis (IPF) with a median survival time only 3 to 5 years¹⁻³. Targeting both increased fibroblast proliferation and myofibroblast differentiation has been considered as a therapeutic strategy in IPF management; therefore, development of agents capable of eradicating myofibroblasts or limiting their genesis is urgently needed. In the last two decades, the vast majority of therapeutics developed for IPF focused on anti-inflammatory, rather than anti-fibrotic effects, and therefore had limited success in the clinic, with the nonspecific suppression of the inflammatory response and potent immunosuppression being the primary obstacles. In 2014, the US Food and Drug Administration (FDA) approved two novel therapeutic agents, pirfenidone and nintedanib, for IPF, each at a cost of almost $100,000 per patient per year. Due to intolerable adverse effects, some IPF patients have switched to nintedanib after discontinuation of pirfenidone 4. Nintedanib, a potent multikinase inhibitor, shows anti-fibrotic and anti-inflammatory effects via blocking several key receptor tyrosine kinases including platelet-derived growth factor (PDGF) receptor, fibroblast growth factor (FGF) receptor, and vascular endothelial growth factor (VEGF) receptor^{5, 6}. Unfortunately, the transforming growth factor-beta (TGF-0) pathway, an important determinant in IPF progression^{7,8}, is not the major target of this drug. In addition, adverse effects are common with nintedanib therapy and worse with the higher dose, resulting in drug discontinuation^{9, 10}. For these reasons, there is an urgent need to seek new and better therapeutics for those diagnosed with IPF. The central idea of this disclosure is to develop effective approaches for selectively targeting fibrogenic pathways without the disturbance of the immune and inflammatory responses and also improving the efficacy of nintedanib treatment. Additionally, Applicant evaluated the antifibrotic properties of the compounds in the phase of established fibrosis rather than in the early period of inflammation. The use of candidate treatments in the “fibrotic” phase of the animal model, which better reflects human IPF, is greatly needed in order to reveal beneficial antifibrotic compounds.

Applicant found that the major protein kinase C substrate MARCKS (myristoylated alanine-rich C kinase substrate) is a potential target molecule for IPF and developed novel peptide-based therapeutics for selective ablation of activated fibroblasts and myofibroblasts without adversely affecting normal fibroblasts. In addition to being a major substrate for protein kinase C, MARCKS is also a phosphatidylinositol 4,5-bisphosphate (PIP2)-associated protein through its phosphorylation site domain (PSD; also known as the basic effector domain) binding to the cell membrane. Phosphorylation by PKC within the MARCKS PSD (Ser159 and Ser163) enhances phosphorylated MARCKS (phospho-MARCKS) detachment from membrane and suppresses the PIP2-sequestering effect^{11, 12} Recent studies have indicated that an important function of the MARCKS PSD, upon phosphorylation, is to provide PI3K with PIP2 pools for PIP3 (phosphatidylinositol (3,4,5)-trisphosphate) production, thereby activating AKT¹³⁻¹⁵ Withholding PIP2 from its enzymes by targeting phospho-MARCKS prevents aberrant production of PIP3, inositol trisphosphate, and diacylglycerol in dysregulated cells but has no effect on enzyme activity in normal cellular processes; this indicates that MARCKS itself may therefore be a more effective target. Based on the sequence of the MARCKS PSD, Applicant have identified a 25-mer peptide, the MPS peptide, which targets the MARCKS PSD Sequence and inhibits AKT activation in cancers ^{14, 16} Based on the findings, a series of small MPS peptides, ranging from 12 to 25 amino acids designed to mimic both the membrane curvature and PIP2 retention activities of MARCKS' PSD/ED motif sequence, have been developed. Their inhibitory efficacy, which is based on PIP2 and PIP3 retention activity, has been tested in the suppression of bleomycin-induced mouse lung fibrosis model in vivo, and in the inhibition of myofibroblast differentiation in vitro, as well as the growth of IPF tissue-derived fibroblasts ex vivo. Below are the results regarding this disclosure.

### Aberrant Elevation of MARCKS Phosphorylation and its Relevance to IPF Fibroblasts

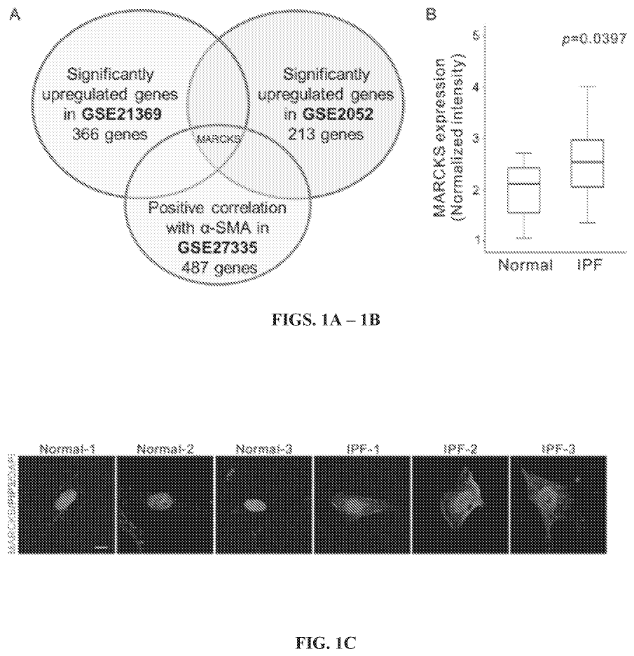

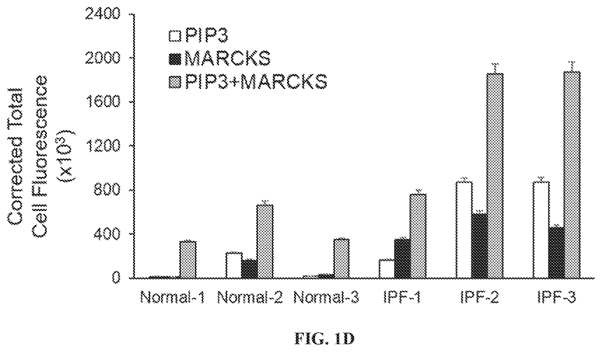

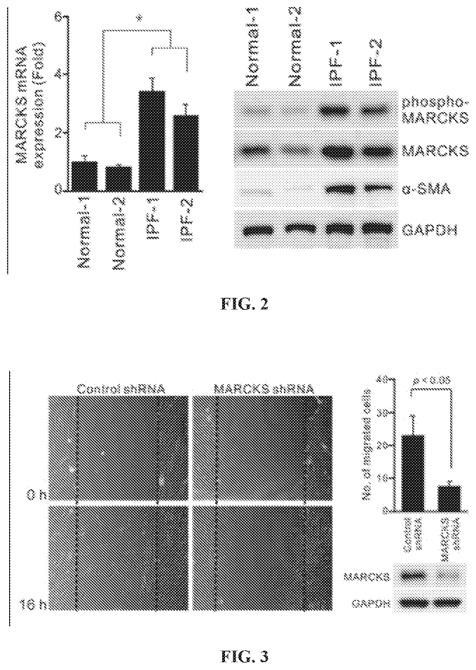

To uncover the regulatory molecules that drive gene expression representative of IPF features in lung fibroblasts, a comparison approach was used in which two different microarray datasets (GSE21369 and GSE2052) were integrated to find genes that are specifically upregulated in lung fibroblasts isolated from IPF patients, as compared to normal fibroblasts from non-IPF patients. Currently, the most definitive molecular marker of the myofibroblast is alpha smooth muscle actin (α-SMA), which is indicative of fibroblast activation and plays a critical role in development and progression of IPF^{17, 18}. Notably, Applicant identified a cluster of 487 genes that were positively correlated with α-SMA expression in dataset GSE27335, which includes profiling data of lung myofibroblast-like cells. By analyzing overlapping genes with the calculated 366 genes in GSE21369 and 213 genes in GSE2052 that were significantly upregulated compared to normal fibroblasts, a panel of 14 genes as candidate targets for controlling fibroblast activation in IPF were identified. In light of the fact that more than a third of all known biomarkers and more than two-thirds of potential disease targets are membrane-related proteins^{19,20}, the critical PIP2-binding partner MARCKS²¹, one of the 14 identified genes, attracted attention and was selected for further study (FIG. 1A). Through the analysis of the transcriptome dataset²², Applicant compared MARCKS gene expression between 13 samples obtained from surgical remnants of biopsies or lungs explanted from patients with IPF that underwent pulmonary transplant and 11 normal histology lung samples resected from patients with lung cancer. A significant elevation of MARCKS expression in IPF lung tissues was observed (FIG. 1B). To validate that MARCKS is dysregulated in IPF fibroblasts, MARCKS expression and its phosphorylation in primary lung fibroblast cells isolated from IPF and non-IPF patients was examined. FIG. 2 shows higher expression of α-SMA, MARCKS and MARCKS phosphorylation at Ser159 and Ser163 (phospho-MARCKS) in two IPF fibroblast cells (IPF-1 and -2) as compared to normal fibroblasts (normal-1 and -2), suggesting the implications of high phospho-MARCKS and MARCKS expression in IPF fibroblasts. Next, a MARCKS-specific short hairpin RNA (MARCKS shRNA) was used to eliminate both phospho-MARCKS and MARCKS expression and showed a 2.9-fold reduction in migration of MARCKS-knockdown cells (FIG. 3). Applicant previously developed a cell-permeable peptide, the MPS peptide, which targets the [symbol] ARCKS [symbol] hosphorylation [symbol] ite domain (PSD; also known as the basic effector domain) and inhibits phospho-MARCKS levels in cancers^{14, 16} As expected, treatment with this peptide in primary IPF fibroblast cells confirmed that MARCKS inhibition reduces cell motility and proliferation (FIG. 4), consistent with shRNA knockdown of MARCKS. These results suggest that MARCKS plays an important role in several phenotypes relevant to IPF. Since MARCKS' function depends on its phosphorylation, Applicant next confirmed phospho-MARCKS levels immune-histologically in both normal lung samples and IPF lung tissues from patients (n=18) receiving or not receiving nintedanib treatment. Immunohistochemical (IHC) analysis of MARCKS phosphorylation showed an increase of phospho-MARCKS signals in the tissue sections from IPF patients (FIG. 5). Strong phospho-MARCKS staining was also observed in tissues from IPF patients undergoing nintedanib therapy. In the fibroblastic foci, it was observed that some of the fibroblast-like cells did not have much immunostaining while some undefined cells displayed strong phospho-MARCKS signal. Presently, it is presumed that the undefined parenchymal cells to be myofibroblasts; a confirmation of this hypothesis can be obtained by performing dual staining of phospho-MARCKS with α-SMA, a myofibroblast marker.

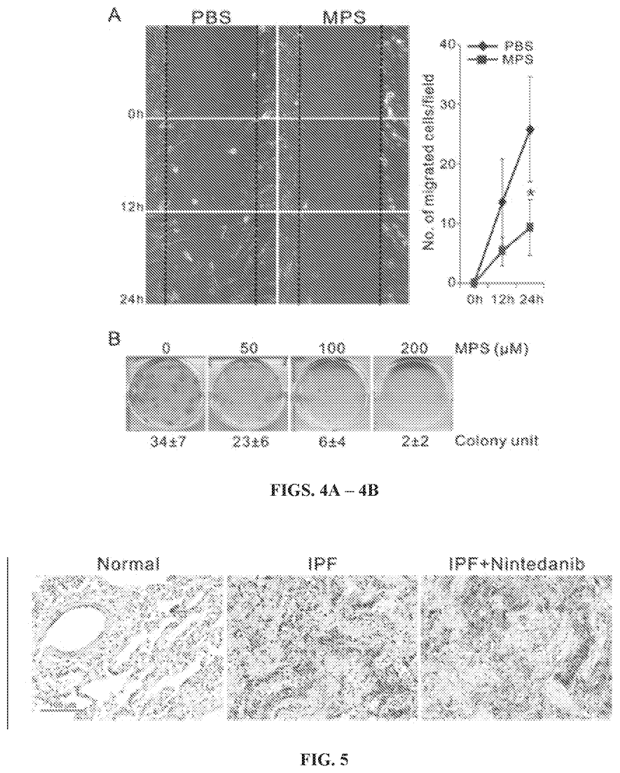

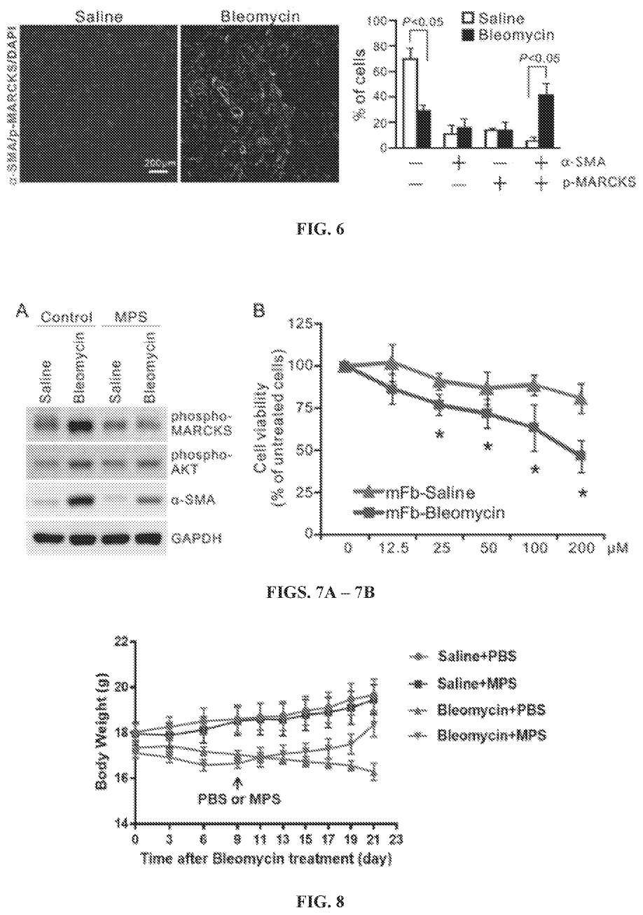

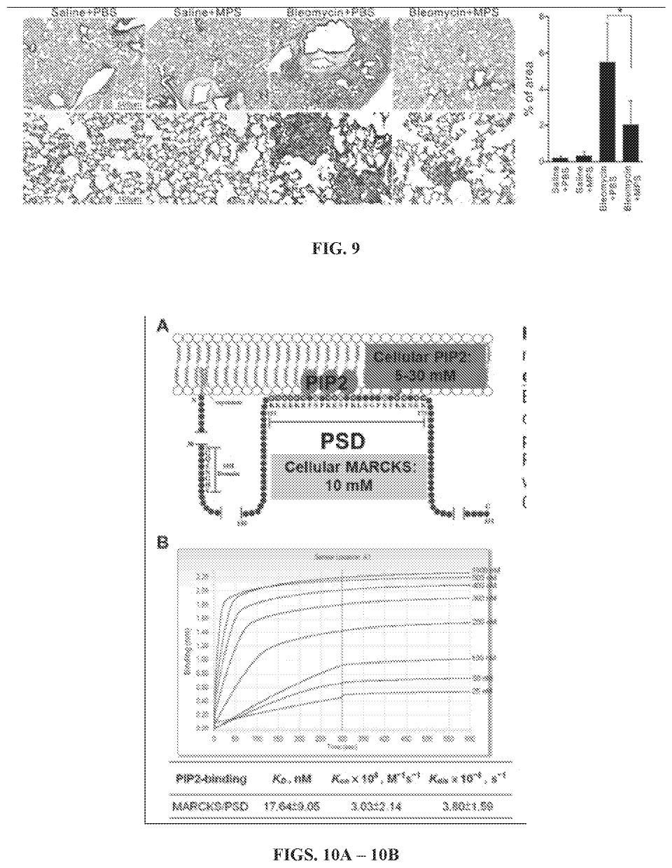

MPS peptide potentially serves as an antifibrotic agent in bleomycin-induced pulmonary fibrosis. Bleomycin remains the standard agent for induction of experimental pulmonary fibrosis in animals²³. Thus, 8-week-old female C57BL/6J mice received saline or bleomycin intratracheally (33 μg in 50 ml of saline) as previously described²³. Lung specimens from bleomycin- or saline-treated mice were collected and subjected to immunofluorescence staining. Elevated co-expression of phospho-MARCKS and α-SMA was seen in bleomycin-treated lung tissues (FIG. 6). Next, lung fibroblast cells isolated from saline- or bleomycin-treated mice (two mice fibroblast cell lines were gifts from Dr. Sem H. Phan, University of Michigan School of Medicine, MI) were incubated with either 100 μM control or MPS peptide for 48 hours. Fibroblasts from bleomycin-treated mice exhibited a decrease in phospho-MARCKS, phospho-AKT and α-SMA expression in the presence of MPS (FIG. 7A). Moreover, MTT assays confirmed that MPS treatment is very effective in decreasing cell viability of these fibroblast cells, as compared to the treatment of fibroblast cells from saline-treated mice (FIG. 7B). The feasibility of the MPS peptide as an antifibrotic agent in a bleomycin-induced pulmonary fibrosis was tested. Upon bleomycin exposure for 9 days, the body weight of mice was obviously decreased, as compared to mice receiving saline (control group). Saline- and bleomycin-exposed mice then were treated with either PBS or MPS peptide (28 mg/kg) intraperitoneally every other day. To ascertain the therapeutic effect of MPS peptide on pulmonary fibrosis, MPS was administered intraperitoneally during the “fibrotic” phase of the model. In total, there were four groups (five mice per group): 1) saline plus PBS; 2) saline plus MPS; 3) bleomycin plus PBS; 4) bleomycin plus MPS. Surprisingly, Applicant observed a continued loss of body weight in the mice exposed to bleomycin plus PBS, but not in the bleomycin-exposed mice with MPS treatment (FIG. 8). After 22 days of bleomycin exposure, mice lungs were collected and processed for histology and Masson's trichrome staining. Bleomycin-exposed mice showed extensive structural changes in the lungs, whereas decreases of fibroblastic lesions and deposited extracellular matrix were seen in the lungs from mice with bleomycin exposure and MPS treatment (FIG. 9). These results suggest that phospho-MARCKS may be a therapeutic target for pulmonary fibrosis.

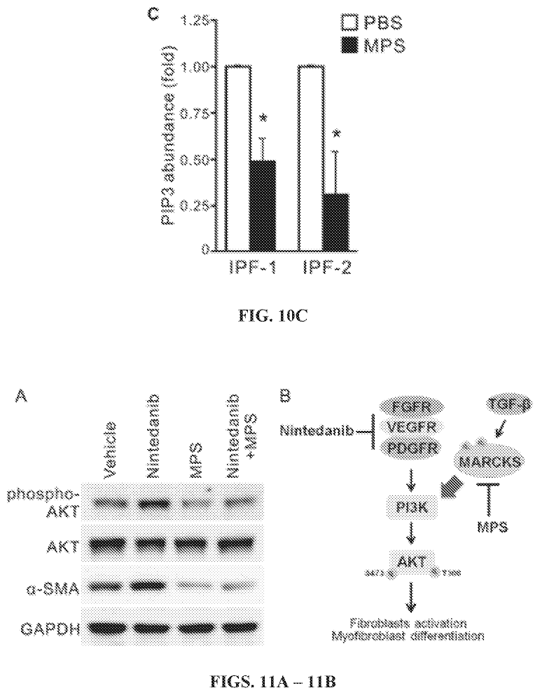

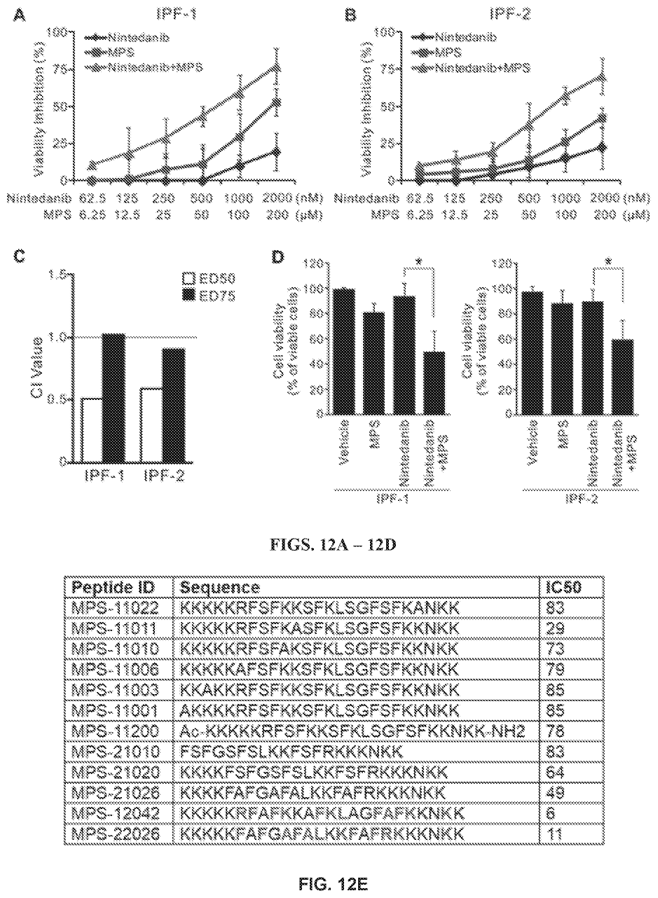

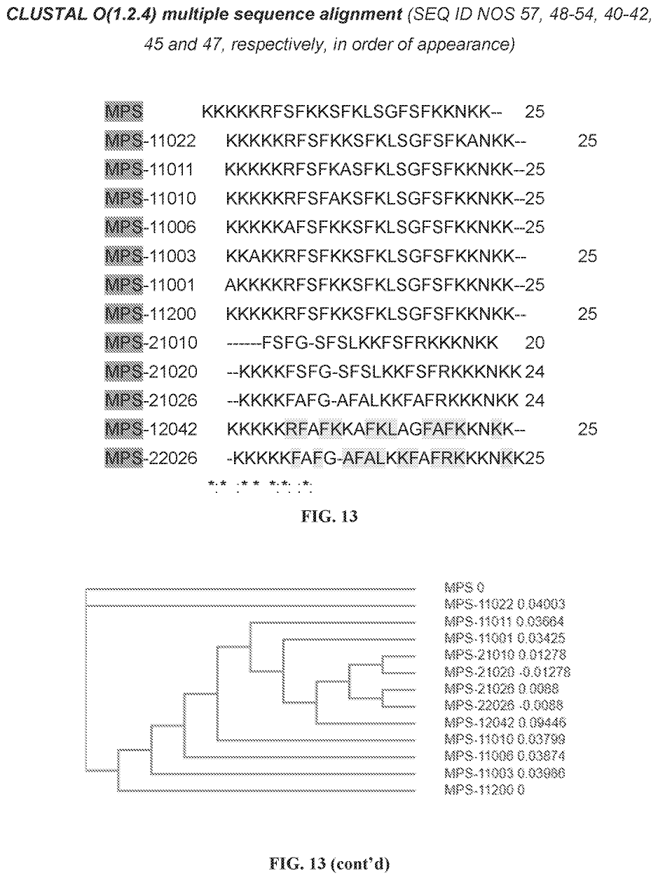

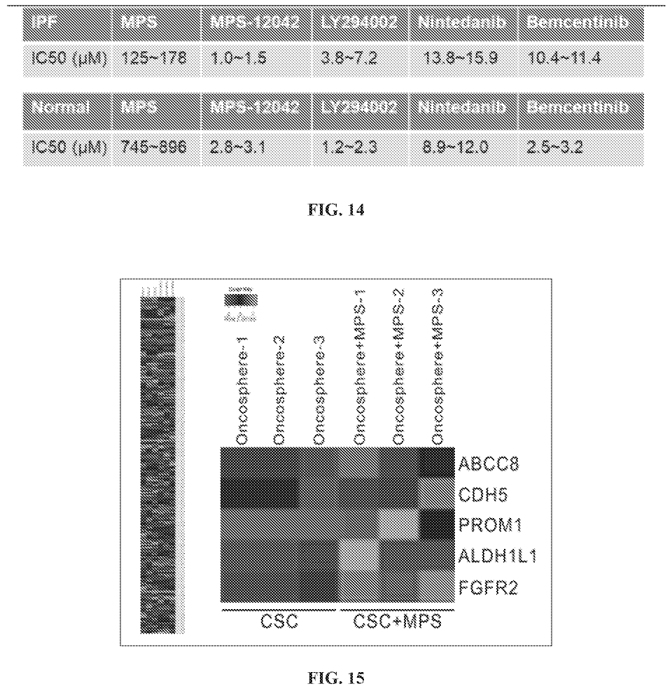

Molecular basis of MPS peptide and its potential for increasing nintedanib efficacy. Given the importance of the PSD in the functionality of MARCKS protein, Applicant previously designed a 25-mer MPS peptide to mimic the MARCKS PSD and found that this peptide can directly inhibit phospho-MARCKS-mediated functions in cancers, while having no cytotoxic effect on normal human epithelial cells^{14, 16} On the basis of the PIP2-binding motif on the MARCKS PSD (FIG. 10A), the effect of this peptide on PIP2 binding and PIP3 synthesis which are the two major determinants for AKT activation was tested¹⁵. A kinetic assay confirmed that this peptide binds PIP2 with a dissociation constant of 17.64 nM (FIG. 10B). As expected, a decrease of PIP3 pools in whole cell lysates of MPS-treated IPF fibroblasts was observed (FIG. 10C), supporting the notion that MPS peptide is able to inhibit AKT activation through trapping PIP2. In view of adverse effects of the current IPF therapeutic nintedanib^{9, 10}, there is an urgent clinical need to improve the efficacy of such treatment in IPF. Since the TGF-β receptor is not a direct target of nintedanib, targeting an element of TGF-β signaling in tandem with nintedanib administration circumvents the shortcomings of nintedanib monotherapy. Given that strong phospho-MARCKS staining was seen in lung tissues from IPF patients with nintedanib therapy (FIG. 5), suggesting that MARCKS is still active under treatment with this multikinase inhibitor. FIG. 11A shows an increase of α-SMA expression upon nintedanib treatment, in agreement with the recent report that nintedanib induces α-SMA, albeit TGF-β signaling was partially affected by high doses of nintedanib treatment²⁴. Surprisingly, there was no change in phospho-AKT after nintedanib treatment. Based on the above observations, it is assumed that TGF-β-directed phospho-MARCKS is a bypass mechanism of activating PI3K/AKT signaling (FIG. 11B); therefore, it seems reasonable that MARCKS inhibition by MPS treatment may improve nintedanib efficacy, permitting lower doses of nintedanib to be used. To this end, the possibility of a synergistic interaction between MPS and nintedanib in order to circumvent the shortcomings of nintedanib monotherapy was tested. Cell viability was decreased in primary IPF fibroblasts when treated with eithernintedanib, MPS peptide, or the combination of nintedanib and MPS peptide, with the greatest inhibition of viability observed in the combination group (FIG. 12A-B). Furthermore, the Chou and Talalay CI (combination index) method²⁵ was used to evaluate the therapeutic interactions between nintedanib and MPS peptide. The addition of MPS substantially enhanced the viability suppression of nintedanib with CI value approximately 0.5 at ED50 (CI<1), indicating the synergistic effects of drug combination (FIG. 12C). Particularly, the values were lower than 1 at ED50, approximately 1 at ED75, and above 1 at ED90 (data not shown). Thus, the combination effect was dose-dependently correlated with the components, and therefore low dose nintedanib in combination with low dose of MPS presents a synergistic effect on cell proliferation. Simultaneously, data from trypan blue exclusion test indicated that cell survival was significantly lower with the combination treatment as compared to control, MPS, and nintedanib (FIG. 12D). On the basis of the sequences of MPS peptide, the rearrangement of PIP2-binding sites in this peptide were designed and synthesized with the intention of enhancing the efficacy and stability of the MPS peptide. FIG. 13 lists the sequences of various MPS derivatives. In light of the cleavage sites of various proteases and PIP2 binding motifs, Applicant replaced some L-isoform amino acids with D-isoform and these peptides were named as MPS-12042 and MPS-22026. To further validate the efficacy of the above MPS derivatives, Applicant performed a dose-course analysis of H1650 cells undergoing each MPS derivative treatment. MTT assays showed IC50 values for various MPS-related peptides (FIG. 13). Since MPS-12042 showed the most effective at killing the highly proliferative cells, H1650, its role in treating IPF fibroblast cells was determined. Using a MTS assay, Applicant found that MPS-12042 treatment has a better efficacy in inhibiting IPF fibroblast proliferation (IC50:1.0-1.5 μM) as compared to MPS peptide (IC50: 125-178 μM). Of note, the concentration at 1 μM remarkably decreased cell proliferation by 50% in IPF fibroblast but not in normal fibroblasts (FIG. 14). In addition to targeting selectivity of MPS-12042, the IC50 for MPS-12042 is lower than the current FDA-approved IPF drug nintedanib (IC50: 13.8-15.9 μM).

As suggested by this disclosure's data, phospho-MARCKS acts as a specific marker for activated fibroblasts, inhibiting MARCKS activity by the use of the MPS peptides could lead to future clinical testing and a potential new therapeutic for IPF patients. The therapeutic potential of the MPS peptide in bleomycin-induced pulmonary fibrosis has demonstrated for the first time and will help to develop treatments that destroy activated fibroblasts and/or myofibroblast without adversely affecting quiescent fibroblasts. In sum, Applicant's studies potentially define and validate therapeutic targets and/or biomarkers for IPF, which may lead to the development of much needed novel therapeutic approaches for IPF.

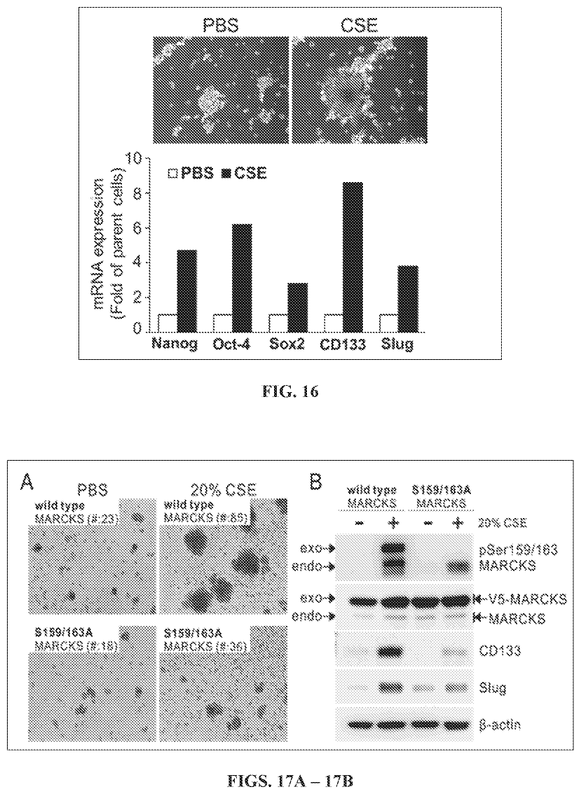

Targeting the MARCKS PSD is associated with inhibition of stem-like cell properties. In light of the importance of the PSD in the functionality of MARCKS protein, Applicant designed a 25-mer MPS peptide to mimic the MARCKS phosphorylation site domain (PSD). A great number of studies have revealed that this 25-mer peptide electrostatically interacts with the plasma membrane. Applicant has found that MPS treatment can directly inhibit the in vitro and in vivo functions of phospho-MARCKS in lung and kidney cancer, while this peptide has no cytotoxic effect on normal human epithelial cells^{14,16} Since phospho-MARCKS drives the progression of lung cancer toward more malignancy²⁹ and cancer stem-like cells (CSCs) participate in cancer malignancy, there may be an association between higher phospho-MARCKS and cancer stemness. Applicant's initial studies have shown that elevation of phospho-MARCKS in lung cancer spheres acted in parallel with increased stemness markers, such as CD133, Oct3/4, SOX2 and Nanog (data not included). The oncospheres were derived from high MARCKS-expressing lung cancer cell lines (H1975 and CL1-5) and primary lung cancer cells (LG704 and LC3: pleural effusion cells isolated from patients with advanced stage) in non-adherent serum-free culture conditions as described previously³⁰⁻³². Flow cytometry confirmed that ˜80% of LG704 oncosphere cells are CD133-positive, a major lung CSCs marker. Culturing these cells in spheroid conditions showed not only more resistance to both DNA damaging agents and EGFR inhibitors but also high tumorigenicity in vivo, as compared to cells in adherent conditions (data not included). Through a comparison of transcriptome profiling between PBS- and MPS-treated LG704 oncospheres by RNA-seq, Applicant identified a total of 352 coding genes altered by MARCKS inhibition (FIG. 15, left). Several of the expected cancer stemness genes were decreased after 50 μM MPS treatment, notably ABCC8, CDH5, PROM1 (CD133), ALDH1L1 and FGFR2 (FIG. 15, right). As sphere formation (or sphere-forming ability) is an indicator of tumor aggressiveness and correlates with poor survival in cancer patients, applicant next confirmed the fact that long-term exposure to smoke potentiates cancer stemness (sphere formation)³³⁻⁴⁴. The sphere-forming ability was assessed by counting the number and size of tumor spheres (oncospheres) under a microscope. Serum-free medium and non-adherent culture conditions were used to culture and enrich the cancer stem-like population from low-invasive lung cancer cell line, CL1-0 cells, which were originally cultured under an adhering culture condition. With non-adherent serum-free culture conditions for seven days of exposure to PBS or cigarette smoke extract (CSE), smoke-treated cells displayed higher oncosphere-forming ability (FIG. 16, top) and elevated expression of various CSC-associated transcriptional factors (FIG. 16, bottom). Furthermore, V5-tagged wild-type and PSD-mutated (S159/163A) MARCKS constructs were introduced into low MARCKS-expressing cells. An approximate 3.7-fold increase in sphere-forming ability in smoke-treated cells with ectopic expression of V5-tagged wild type MARCKS was observed, whereas smoke-enhanced sphere-forming ability and stemness gene expression were not obviously seen in cells with overexpression of phosphorylation-defective S159/163A MARCKS (FIG. 17). Pharmacologically, Applicant treated smoke-enriched oncospheres derived from H292 cells with MPS peptide to target the MARCKS PSD. FIG. 18 shows inhibitory effects of the MPS peptide on the number and size of oncospheres as well as the expression of stemness genes. Such inhibition of cancer stemness by MPS peptide may be attributed to the suppression of tobacco smoke-induced MARCKS phosphorylation.

### Cell Culture

Human primary fibroblast cells were obtained from airway tissues provided from the UC Davis Medical Hospital (Sacramento, Calif.) with consent. The protocol for human tissue procurement and usage were periodically reviewed and approved by the University Human Subject Research Review Committee. Primary fibroblast cell lines, IPF-1 and IPF-2 cells, were established from IPF patients. Cells were obtained from lung biopsies and the diagnosis of IPF was supported by patient history, physical examination, pulmonary function tests, and typical high-resolution chest computed tomography findings of IPF. In all cases, the diagnosis of IPF was confirmed by microscopic analysis of lung tissue and demonstrated the characteristic morphological findings of usual interstitial pneumonia. All patients fulfilled the criteria for the diagnosis of IPF as established by the American Thoracic Society and European Respiratory Society. Non-fibrotic primary control adult human lung fibroblast lines, Normal-1 and Normal-2 cells were used. These lines were established from normal lung tissue or histologically normal lung tissue adjacent to carcinoid tumor. The IPF cell line, LL97A, was purchased from the American Type Culture Collection (ATCC) (Manassas, Va.). Lung fibroblast lines were cultured in high-glucose DMEM or RPMI-1640 medium with 10% fetal bovine serum and 1% penicillin-streptomycin at 37° C. in a humidified atmosphere of 5% CO2. Fibroblasts were used between passages 4 and 8. Cells were characterized as fibroblasts as described²⁶.

### Quantitative Real-Time PCR

The mRNA expression level of target genes was detected by real-time reverse transcription polymerase chain reaction (RT-qPCR) using primers as described in the Primers section below. The house keeping gene TATA-box binding protein (TBP) was used as the reference gene. The relative expression level of target genes compared with that of TBP was defined as −ΔCT=−[CT_{target}−CT_{TBP}]. The target/TBP mRNA ratio was calculated as 2^{-ΔCT}×K, where K is a constant.

### Patient Lung Specimens and Immunohistochemical Staining

IPF lung tissue and non-IPF normal lung specimens were obtained from patients with histologically confirmed IPF who underwent surgical resection at the UC Davis Medical Center. This investigation was approved by the Institutional Review Board of the UC Davis Health System. Written informed consent was obtained from all patients. Formalin-fixed and paraffin-embedded specimens were used, and level of phospho-MARCKS was analyzed by immunohistochemical staining as described previously^{14, 16, 27} These results were also reviewed and scored independently by two pathologists.

### Kinetic Assay

Real-time binding of the peptide mimicking the phosphorylation site domain of MARCKS (MPS peptide, amino acids 151 to 175 from the wild-type MARCKS protein) to biotin-labeled PIP2 was evaluated using biolayer interferometry (BLI) on an Octet RED96 system (ForteBio) following the manufacturer's instructions. Briefly, the ligand, PIP2 labeled with biotin at the sn-1 position (1000 nM in ddH₂O), was immobilized on Super Streptavidin (SSA) biosensors for 10 minutes. A binding assay was performed with the MPS analyte at various concentrations from 0 to 1000 nM in ddH₂O. Association and dissociation were monitored for 5 minutes. Assays were performed at 24° C. Data were analyzed using Octet Data Analysis Software 7.0 (ForteBio).

### PI(3,4,5)P3 Quantitation

Cells were harvested and precipitated by trichloroacetic acid. PIP3 lipids were extracted twice from the trichloroacetic acid precipitated fraction by methanol: chloroform (2:1). After acidification, organic-phase lipids were used for PIP3 quantitation, based on the protocol for the PIP3 Mass ELISA kit (Echelon Biosciences, Salt Lake, Utah). Briefly, the lipid extract from cultured cells was mixed with the PIP3-specific detector protein, which was then incubated in a PIP3-coated microplate for competitive binding. After several washes, the microplate was then incubated with a HRP-linked secondary detector and tetramethylbenzidine substrate for color development. To stop further color development, 2M H₂SO₄ solution was then added. Microplates were read at an absorbance wavelength of 450 nm. A series of different dilutions of PIP3 standards were used for establishing a standard curve for each reaction. Cellular PIP3 amounts could be estimated by comparing the absorbance in the wells with the values in the standard curve. Experiments were conducted in triplicate dishes and repeated in two independent cultures with cell density 5×10⁶ cells/100-mm dish.

### Transwell Migration Assays

An in vitro cell migration assay was performed as previously described^{13, 14} using Transwell chambers (8-μm pore size; Costar, Cambridge, Mass.). Briefly, 2×10⁴ cells were seeded on top of the polycarbonate filters, and 0.5 ml of growth medium with scrambled or MPS peptide (100 μM) was added to both the upper and lower wells. After incubation for 20 hours, filters were swabbed with a cotton swab, fixed with methanol, and then stained with Giemsa solution (Sigma). The cells attached to the lower surface of the filter were counted under a light microscope (10× magnification).

### Scratch Wound-Healing Assay

Cells were seeded to six-well tissue culture dishes and grown to confluence. A linear wound was introduced to each confluent monolayer using a pipette tip and washed three times with PBS. Thereafter, cell morphology and migration were observed and photographed at regular intervals for 12 and 24 hours. The number of cells migrating into the cell-free zone was acquired under a light microscope and counted.

### Immunoblotting and Immunofluorescent Staining

Western blot analyses and the preparations of whole-cell lysates have been previously described^{14, 16, 27} For whole cell lysates, cells were lysed in a lysis buffer (50 mM Tris-HCl (pH 7.4), 1% Triton X-100, 10% glycerol, 150 mM NaCl, 1 mM EDTA, 20 μg/ml leupeptin, 1 mM PMSF and 20 μg/ml aprotinin) and separated by SDS-PAGE. Immunoblotting was conducted with appropriate antibodies followed by chemiluminescent detection. For immunofluorescent staining, after an antigen retrieval step, tissue slides were reacted with antibodies against FITC-labeled α-SMA and TRITC-conjugated phospho-MARCKS, and nuclei were demarcated with DAPI staining. The cells were mounted onto slides and visualized using fluorescence microscopy (model Axiovert 100; Carl Zeiss, Oberkochen, Germany) or a Zeiss LSM510 laser-scanning confocal microscope image system.

### Bleomycin-induced Lung Fibrosis

Female C57BL/6J mice (8-week-old) were purchased from Jackson Laboratory (Sacramento, Calif.) and receive saline or bleomycin intratracheally as previously described²³. Briefly, mice were anesthetized with 5% isoflurane and administered bleomycin (APP Pharmaceuticals, Schaumburg, Ill.) at a dose of 0.005 U/g mouse via intratracheal aspiration on day 0. Control animals received an equal volume of sterile saline only. In early fibrogenic phase, these mice were intraperitoneally (i.p) injected with either PBS, or MPS peptide (28 mg/kg) every two days. At 21 days of bleomycin insult, these mice were sacrificed and the lungs were collected for histological analysis. Mouse experiments were approved by the Institutional Animal Care and Use Committee of UC Davis.

### Cell Proliferation and Colony Formation Assays

Cells were seeded onto 96-well plates at a density of 5-10×103 cells per well and cultured for the indicated treatment. Cell proliferation was evaluated using a MTS assay kit (Promega, Madison, Wis.). Twenty microliters of the combined MTS/PMS solution was added into each well, incubated for 3 hours at 37° C., and the absorbance was measured at 490 nm by using an ELISA reader. For Trypan blue test, cells were plated on 12-well plates and treated with the indicated chemotherapeutic agents. After 72 hours, both attached and detached cells were collected and then stained with 0.2% trypan blue (0.1% final concentration), and the number of trypan blue-positive and -negative cells was counted using a haemocytometer under low-power microscopy. For colony-forming assays, 200 cells were seeded in each well of six-well plates. IPF-1 or IPF-2 primary cells were treated with peptides at the indicated concentrations for 10 days. Colonies were stained using 0.001% crystal violet and the number of colonies with a diameter greater than 0.5 mm was counted under an inverted microscope.

### Reagents and Antibodies

Dulbecco's Modified Eagle's medium, RPMI-1640 medium, fetal bovine serum and penicillin-streptomycin were purchased from Life Technologies Inc. (Carlsbad, Calif.). Lipofect-AMINE™ was purchased from Invitrogen (Carlsbad, Calif.). VECTASTAIN® Elite ABC Kit (Rabbit IgG), VECTOR® Hematoxylin QS nuclear counterstain and DAB solution were purchased from VECTOR Laboratories Inc. (Burlingame, Calif.). Anti-pSer158 MARCKS (clone EP2113Y) and anti-MARCKS (clone EP1446Y) were purchased from Abcam (Cambridge, Mass.). Anti-pSer159/163 MARCKS (clone D13D2), anti-pSer473 AKT, anti-pSer308 AKT, anti-AKT, anti-α-SMA, anti-GAPDH and anti-3-actin antibodies were purchased from Cell Signaling Technology, Inc. (Danvers, Mass.).

### Primers

The all primers for quantitative real-time PCR used were as follows: the α-SMA forward primer 5′-TCCTCATCCTCCCTTGAGAA-3′ (SEQ ID NO: 60) and the reverse primer 5′-ATGAAGGATGGCTGGAACAG-3′ (SEQ ID NO: 61); the COL1A1 forward primer 5′-ACGAAGACATCCCACCAATCACCT-3′ (SEQ ID NO: 62) and the reverse primer 5′-AGATCACGTCATCGCACAACACCT-3′ (SEQ ID NO: 63); the THY1 forward primer 5′-AGAGACTTGGATGAGGAG-3′ (SEQ ID NO: 64) and the reverse primer 5′-CTGAGAATGCTGGAGATG-3′ (SEQ ID NO: 65); the FN1 forward primer 5′-TCCACAAGCGTCATGAAGAG-3′ (SEQ ID NO: 66) and the reverse primer 5′-CTCTGAATCCTGGCATTGGT-3′ (SEQ ID NO: 67); the VIM forward primer 5′-AACTTCTCAGCATCACGATGAC-3′ (SEQ ID NO: 68) and the reverse primer 5′-TTGTAGGAGTGTCGGTTGTTAAG-3′ (SEQ ID NO: 69); the MARCKS forward primer 5′-TTGTTGAAGAAGCCAGCATGGGTG-3′ (SEQ ID NO: 70) and the reverse primer 5′-TTACCTTCACGTGGCCATTCTCCT-3′ (SEQ ID NO: 71).

### Patient Lung Specimens and Immunohistochemical Staining

IPF lung tissue and non-IPF normal lung specimens were obtained from patients with histologically confirmed IPF who underwent surgical resection at the UC Davis Medical Center. This investigation was approved by the Institutional Review Board of the UC Davis Health System. Written informed consent was obtained from all patients. Formalin-fixed and paraffin-embedded specimens were used, and level of phospho-MARCKS was analyzed by immunohistochemical staining as described previously 1. Detailed experimental procedures were modified from the paraffin immunohistochemistry protocol supplied by the manufacturer (Cell Signaling, Danvers, Mass.). The slides were de-paraffinized in xylene and rehydrated in graded alcohol and water. An antigen retrieval step (10 nM sodium citrate (pH 6.0) at a sub-boiling temperature) was used for each primary antibody. Endogenous peroxidase activity was blocked by 3% hydrogen peroxide followed by blocking serum and incubation with appropriate antibodies overnight at 4° C. Detection of immunostaining was carried out by using the VECTASTAIN® ABC system, according to the manufacturer's instructions (Vector Laboratories, Burlingame, Calif.). A four-point staining intensity scoring system was devised to confirm the relative expression of phospho-MARCKS in lung specimens; scores ranged from zero (no expression) to 3 (highest-intensity staining) as described previously^{14,27-29}. The results were classified into two groups according to the intensity and extent of staining: in the low-expression group, staining was observed in 0-1% of the cells (staining intensity score=0), in less than 10% of the cells (staining intensity score=1), or in 10%-25% of the cells (staining intensity score=2); in the high-expression group, staining was present more than 25% of the cells (staining intensity score=3).

### Experiment No. 2

### Tackling the MARCKS-PIP3 Circuit to Attenuate Chronic Pulmonary Fibrosis

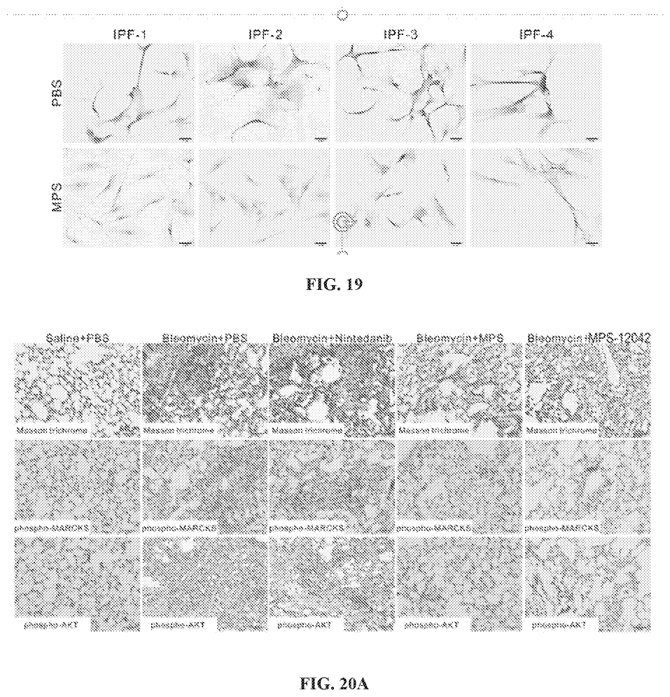

As noted in Experiment No. 1, Applicant found that MARCKS expression as well as MARCKS phosphorylation (phospho-MARCKS) are elevated in IPF tissues and cells. This demonstrated that this phenomena was observed in both in-vitro as well as in-vivo in the bleomycin mouse model of pulmonary fibrosis. MARCKS levels and activity (phospho-MARCKS) were correlated with higher pro-fibrotic activity including cell proliferation, extracellular matrix production, invasiveness, and fibroblast differentiation. Upon treatment with MPS peptides, which target MARCKS activity, Applicant observes attentuation of these activities. The second significant finding was that MARCKS mediates these profibrotic effects through through the PI3K/AKT pathway. Applicant demonstrated that this signaling pathway was upregulated in both IPF tissue and cells as well as in the bleomycin mouse model. Targeting of these activities with MPS peptide results in decreased AKT activity and downstream pro-fibrotic signals. The mechanism through which this occurs is through regulation of PIP2 availability at the cell membrane. In an unphosprylated state, MARCKS is able to bind PIP2 at the cell membrane, preventing PI3K proteins from converting PIP2 into PIP3 and effecting downstream AKT activity. Upon phosphorylation, MARCKS is released from the cell membrane into the cytosol, freeing up PIP2 and allowing PI3K to convert PIP2 into PIP3. In order to show that elevated MARCKS activity and levels are correlated with elevated levels of PIP3, Applicant stained IPF lung fibroblast cells and normal lung fibroblast cells and subjected them to confocal microscopy. Applicant demonstrates in FIGS. 1C and 1D that PIP3 and MARCKS levels are elevated in IPF lung fibroblast cells compared to normal lung fibroblast cells and that high levels of MARCKS is correlated with higher levels of PIP3. Applicant also demonstrates that higher PIP3 is observed in IPF lung fibroblast cells and that PIP3 levels are reduced after MPS treatment in FIG. 19. Applicant obtained multiple IPF lung fibroblast cells and treated them with either PBS or 100 μM MPS peptide for 12 hours and subjected to immunocytochemistry utilizing anti-PIP3 antibody. Applicant demonstrates that higher PIP3 levels are observed in IPF lung fibroblast cells and levels are reduced after MPS peptide treatment.

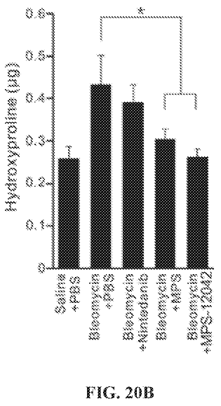

Additionally, Applicant also modified the MPS peptide to improve the stability and potency of the peptide. Of note, MPS-12042 demonstrated marked improvement in potency. Applicant tested this peptide against currently approved IPF therapeutic, nintedanib, as well as MPS peptide in the belomycin mouse model of pulmonary fibrosis. As shown in FIG. 20, MPS-12042 has superior efficacy at attenuating phospho-MARCKS and phospho-AKT as well as reducing overall fibrosis and extracellular matrix deposition in mouse lungs exposed to bleomycin.

In all, these additional pieces of evidence demonstrate the role of MARCKS in regulating PIP2/PI3K/PIP3/AKT activity and that MPS peptides are a potential and viable option to target these activities in IPF.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### PARTIAL SEQUENCE LISTING

Bolded amino acids are D-isoforms.

(WS-21010)
SEQ ID NO: 40
FSFGSFSLKKFSFRKKKNKK
(WS-21020)
SEQ ID NO: 41
KKKKFSFGSFSLKKFSFRKKKNKK
(WS-21026)
SEQ ID NO: 42
KKKKFAFGAFALKKFAFRKKKNKK
(WS-31010)
SEQ ID NO: 43
KKKNKSFFGKSKKFKKKKSF
(WS-31020)
SEQ ID NO: 44
KRFLSKKKNKSFFGKSKKFKKKKSF
(WS-12042)
SEQ ID NO: 45
KKKKKRFAFKKAFKLAGFAFKKNKK
(MPS-12041)
SEQ ID NO: 46
KKKKKRFAFKKAFKLAGFAFKKNKK
(WS-22026)
SEQ ID NO: 47
KKKKKFAFGAFALKKFAFRKKKNKK
(WS-11022)
SEQ ID NO: 48
KKKKKRFSFKKSFKLSGFSFKANKK
(MPS-11011)
SEQ ID NO: 49
KKKKKRFSFKASFKLSGFSFKKNKK
(WS-11010)
SEQ ID NO: 50
KKKKKRFSFAKSFKLSGFSFKKNKK
(WS-11006)
SEQ ID NO: 51
KKKKKAFSFKKSFKLSGFSFKKNKK
(WS-11003)
SEQ ID NO: 52
KKAKKRFSFKKSFKLSGFSFKKNKK
(MPS-11001)
SEQ ID NO: 53
AKKKKRFSFKKSFKLSGFSFKKNKK
(WS-11200)
SEQ ID NO: 54
Ac-KKKKKRFSFKKSFKLSGFSFKKNKK-NH2

Bolded amino acids are D-isoforms.

SEQ ID NO: 55 (Consensus)

X(K/R/A)F(A/S)FRX, wherein X is any amino acid.

SEQ ID NO: 56 (Consensus)

X(K/R/A)F(A/S)FRX, wherein one or both X are K (lysine).

SEQ ID NO: 57 (WT MPS)

SEQ ID NO: 58
KKKKKRFSFKKSFKLSGFSFKKNKK

XXXRYAYXXAYX, where X is any amino acid, and Y is a hydrophobic amino acid residue.

SEQ ID NO: 59

XXXXXRYAYXXAYXLAGYAYXXNXX, wherein X is any amino acid, and Y is a hydrophobic amino acid residue.

### REFERENCES

1 Ley B, Collard H R, King T E, Jr. Clinical course and prediction of survival in idiopathic pulmonary fibrosis. American journal of respiratory and critical care medicine 2011; 183:431-440.
2 Nalysnyk L, Cid-Ruzafa J, Rotella P, Esser D. Incidence and prevalence of idiopathic pulmonary fibrosis: review of the literature. European respiratory review: an official journal of the European Respiratory Society 2012; 21:355-361.
3 Martinez F J, Collard H R, Pardo A et al. Idiopathic pulmonary fibrosis. Nature reviews Disease primers 2017; 3:17074.
4 Milger K, Kneidinger N, Neurohr C, Reichenberger F, Behr J. Switching to nintedanib after discontinuation of pirfenidone due to adverse events in IPF. The European respiratory journal 2015; 46:1217-1221.
5 Wollin L, Wex E, Pautsch A et al. Mode of action of nintedanib in the treatment of idiopathic pulmonary fibrosis. The European respiratory journal 2015; 45:1434-1445.
6 Wollin L, Maillet I, Quesniaux V, Holweg A, Ryffel B. Antifibrotic and anti-inflammatory activity of the tyrosine kinase inhibitor nintedanib in experimental models of lung fibrosis. J Pharmacol Exp Ther 2014; 349:209-220.
7 Tatler A L, Jenkins G. TGF-beta activation and lung fibrosis. Proceedings of the American Thoracic Society 2012; 9:130-136.
8 Nakerakanti S, Trojanowska M. The Role of TGF-beta Receptors in Fibrosis. The open rheumatology journal 2012; 6:156-162.
9 Dimitroulis I A. Nintedanib: a novel therapeutic approach for idiopathic pulmonary fibrosis. Respiratory care 2014; 59:1450-1455.
10 Rangarajan S, Locy M L, Luckhardt T R, Thannickal V J. Targeted Therapy for Idiopathic Pulmonary Fibrosis: Where To Now? Drugs 2016; 76:291-300.
11 Gambhir A, Hangyas-Mihalyne G, Zaitseva I et al. Electrostatic sequestration of PIP2 on phospholipid membranes by basic/aromatic regions of proteins. Biophysical journal 2004; 86:2188-2207.
12 McLaughlin S, Murray D. Plasma membrane phosphoinositide organization by protein electrostatics. Nature 2005; 438:605-611.
13 Chen C H, Thai P, Yoneda K, Adler K B, Yang P C, Wu R. A peptide that inhibits function of Myristoylated Alanine-Rich C Kinase Substrate (MARCKS) reduces lung cancer metastasis. Oncogene 2014; 33:3696-3706.
14 Chen C H, Statt S, Chiu C L et al. Targeting myristoylated alanine-rich C kinase substrate phosphorylation site domain in lung cancer. Mechanisms and therapeutic implications. American journal of respiratory and critical care medicine 2014; 190:1127-1138.
15 Ziemba B P, Burke J E, Masson G, Williams R L, Falke J J. Regulation of PI3K by PKC and MARCKS: Single-Molecule Analysis of a Reconstituted Signaling Pathway. Biophysical journal 2016; 110:1811-1825.
16 Chen C H, Fong LWR, Yu E, Wu R, Trott J F, Weiss R H. Upregulation of MARCKS in kidney cancer and its potential as a therapeutic target. Oncogene 2017; 36:3588-3598.
17 Wynn T A. Integrating mechanisms of pulmonary fibrosis. The Journal of experimental medicine 2011; 208:1339-1350.
18 Lepparanta O, Sens C, Salmenkivi K et al. Regulation of TGF-beta storage and activation in the human idiopathic pulmonary fibrosis lung. Cell and tissue research 2012; 348:491-503.
19 Josic D, Clifton J G, Kovac S, Hixson D C. Membrane proteins as diagnostic biomarkers and targets for new therapies. Current opinion in molecular therapeutics 2008; 10:116-123.
20 Hopkins A L, Groom C R. Target analysis: a priori assessment of druggability. Ernst Schering Research Foundation workshop 2003:11-17.
21 Aderem A. Signal transduction and the actin cytoskeleton: the roles of MARCKS and profilin. Trends in biochemical sciences 1992; 17:438-443.
22 Wang X M, Zhang Y, Kim H P et al. Caveolin-1: a critical regulator of lung fibrosis in idiopathic pulmonary fibrosis. The Journal of experimental medicine 2006; 203:2895-2906.
23 Limjunyawong N, Mitzner W, Horton M R. A mouse model of chronic idiopathic pulmonary fibrosis. Physiological reports 2014; 2:e00249.
24 Rangarajan S, Kurundkar A, Kurundkar D et al. Novel Mechanisms for the Antifibrotic Action of Nintedanib. American journal of respiratory cell and molecular biology 2016; 54:51-59.
25 Chou T C, Talalay P. Quantitative analysis of dose-effect relationships: the combined effects of multiple drugs or enzyme inhibitors. Advances in enzyme regulation 1984; 22:27-55.
26 Chen B, Polunovsky V, White J et al. Mesenchymal cells isolated after acute lung injury manifest an enhanced proliferative phenotype. The Journal of clinical investigation 1992; 90:1778-1785.
27 Chen C H, Cheng C T, Yuan Y et al. Elevated MARCKS phosphorylation contributes to unresponsiveness of breast cancer to paclitaxel treatment. Oncotarget 2015; 6:15194-15208.
28 Kuo T C, Tan C T, Chang Y W et al. Angiopoietin-like protein 1 suppresses SLUG to inhibit cancer cell motility. J Clin Invest 2013; 123:1082-1095.
29 Chen C H, Chiu C L, Adler K B, Wu R. A novel predictor of cancer malignancy: up-regulation of myristoylated alanine-rich C kinase substrate phosphorylation in lung cancer. American journal of respiratory and critical care medicine 2014; 189:1002-1004.
30 Eramo, A. et al. Identification and expansion of the tumorigenic lung cancer stem cell population. Cell Death Differ 15, 504-514, doi:4402283 [pii] 10.1038/sj.cdd.4402283 (2008).
31 Noto, A. et al. Stearoyl-CoA desaturase-1 is a key factor for lung cancer-initiating cells. Cell Death Dis 4, e947, doi:10.1038/cddis.2013.444 cddis2013444 [pii] (2013).
32 Nolte, S. M. et al. A cancer stem cell model for studying brain metastases from primary lung cancer. J Natl Cancer Inst 105, 551-562, doi:10.1093/jnci/djt022 djt022 [pii] (2013).
33 An, Y. et al. Cigarette smoke promotes drug resistance and expansion of cancer stem cell-like side population. PloS one 7, e47919, doi:10.1371/journal.pone.0047919 (2012).
34 Perumal, D. et al. Nicotinic acetylcholine receptors induce c-Kit ligand/Stem Cell Factor and promote stemness in an ARRB1/beta-arrestin-1 dependent manner in NSCLC. Oncotarget 5, 10486-10502, doi:10.18632/oncotarget.2395 (2014).
35 Guha, P. et al. Nicotine promotes apoptosis resistance of breast cancer cells and enrichment of side population cells with cancer stem cell-like properties via a signaling cascade involving galectin-3, alpha9 nicotinic acetylcholine receptor and STAT3. Breast Cancer Res Treat 145, 5-22, doi:10.1007/s10549-014-2912-z (2014).
36 Jedrzejas, M., Skowron, K. & Czekaj, P. Stem cell niches exposed to tobacco smoke. Przegl Lek 69, 1063-1073 (2012).
37 Zhang, M. et al. Mithramycin represses basal and cigarette smoke-induced expression of ABCG2 and inhibits stem cell signaling in lung and esophageal cancer cells. Cancer research 72, 4178-4192, doi:10.1158/0008-5472.CAN-11-3983 (2012).
38 Yu, C. C. & Chang, Y. C. Enhancement of cancer stem-like and epithelial-mesenchymal transdifferentiation property in oral epithelial cells with long-term nicotine exposure: reversal by targeting SNAIL. Toxicol Appl Pharmacol 266, 459-469, doi:10.1016/j.taap.2012.11.023 (2013).
39 Pluchino, L. A. & Wang, H. C. Chronic exposure to combined carcinogens enhances breast cell carcinogenesis with mesenchymal and stem-like cell properties. PloS one 9, e108698, doi:10.1371/journal.pone.0108698 (2014).
40 Liu, Y. et al. Epithelial-mesenchymal transition and cancer stem cells, mediated by a long non-coding RNA, HOTAIR, are involved in cell malignant transformation induced by cigarette smoke extract. Toxicol Appl Pharmacol 282, 9-19, doi:10.1016/j.taap.2014.10.022 (2015).
41 Nieh, S. et al. Regulation of tumor progression via the Snail-RKIP signaling pathway by nicotine exposure in head and neck squamous cell carcinoma. Head Neck 37, 1712-1721, doi:10.1002/hed.23820 (2015).
42 Wang, B. et al. Epigenetic silencing of microRNA-218 via EZH2-mediated H3K27 trimethylation is involved in malignant transformation of HBE cells induced by cigarette smoke extract. Arch Toxicol 90, 449-461, doi:10.1007/s00204-014-1435-z (2016).
43 Liu, Y. et al. Tumorigenesis of smoking carcinogen 4-(methylnitrosamino)-1-(3-pyridyl)-1-butanone is related to its ability to stimulate thromboxane synthase and enhance stemness of non-small cell lung cancer stem cells. Cancer Lett 370, 198-206, doi:10.1016/j.canlet.2015.10.017 (2016).
44 Lee, T. Y. et al. Increased chemoresistance via Snail-Raf kinase inhibitor protein signaling in colorectal cancer in response to a nicotine derivative. Oncotarget 7, 23512-23520, doi:10.18632/oncotarget.8049 (2016).

1. An isolated polypeptide comprising an amino acid sequence selected from the group of SEQ ID NOs: 45, 40-56, 58 or 59, or an equivalent of each thereof.

2. The isolated polypeptide of claim 1, wherein the polypeptide comprises the KKKKKRFAFKKAFKLAGFAFKKNKK (SEQ ID NO: 45), or an equivalent thereof.

3. The isolated polypeptide of claim 1, wherein an equivalent comprises a polypeptide having at least 80% sequence identity to the isolated polypeptide of claim 1 or a polypeptide encoded by a polynucleotide that hybridizes to an isolated polynucleotide that encodes the polypeptide of claim 1 or its complement, and the bolded amino acids are substituted with D-amino acids, that are optionally unmodified from the polypeptide of SEQ ID. Nos. 45, 40-56, 58 or 59, respectively.

4. The isolated polypeptide of claim 1, wherein the polypeptide is selected from the group of SEQ ID NOs. 45-47, 58 or 59, or an equivalent thereof.

5. The isolated polypeptide of claim 4, wherein the equivalent comprises a polypeptide having at least 80% sequence identity to the isolated polypeptide of claim 3 or a polypeptide encoded by a polynucleotide that hybridizes to an isolated polynucleotide that encodes the polypeptide of claim 4 or its complement, and wherein the bolded amino acids are substituted with D-amino acids and retains the D-amino acids.

6. The isolated polypeptide of claim 1, wherein the isolated polypeptide comprises no more than 51 amino acids.

7. The isolated polypeptide of claim 1, wherein the isolated polypeptide comprises no more than 35 amino acids.

8. The isolated polypeptide of claim 1, further comprising one or more of: an amino acid sequence to facilitate entry of the isolated polypeptide into the cell, a targeting polypeptide, or a polypeptide that confers stability to the polypeptide.

9. An isolated polynucleotide encoding the isolated polypeptide of claim 1 or the complement thereof.

10. (canceled)

11. An isolated polynucleotide having at least 80% sequence identity to the polynucleotide of claim 9.

12. A vector comprising the isolated polynucleotide of claim 9, and optionally regulatory sequences operatively linked to the isolated polynucleotide for replication and/or expression.

13. The vector of claim 12, wherein the vector is an AAV vector.

14. A host cell comprising one or more of the isolated polypeptide of claim 1.

15. (canceled)

16. A composition comprising a carrier and one or more of the isolated polypeptide of claim 1.

17. (canceled)

18. The composition of claim 16, further comprising a chemotherapeutic agent or drug, or an anti-fibrotic agent or drug.

19. A method of treating disease or disease symptoms associated with fibrosis in a subject in need thereof, comprising administering to the subject an effective amount of one or more of the isolated polypeptide of claim 1, or an equivalent thereof.

20. (canceled)

21. The method of claim 19, further comprising administering an effective amount of an anti-fibrotic agent or drug, that is optionally nintedanib or pirfenidone.

22. A method for one or more of inhibiting cancer cell growth, treating cancer, inhibiting metastasis, inhibiting cancer stem cell growth, inhibiting cancer stemness, inhibiting tumor cell mobility, restoring sensitivity of a resistant cancer cell to a chemotherapeutic agent, in a subject in need thereof, comprising administering to the subject an effective amount of one or more of the isolated polypeptide of claim 1, or an equivalent thereof.

23. (canceled)

24. (canceled)

25. The method of claim 22, further comprising administering to the subject an effective amount of an anti-cancer drug or agent.

26. The method of claim 25, wherein the anti-cancer drug or agent is from the group consisting of a tyrosine kinase inhibitor (TKI) such as EGFR and VEGFR TKIs, a platinum drug or an immunotherapeutic.

27. A method for delivering a polypeptide of, or an equivalent thereof across the blood brain barrier in a subject in need thereof comprising administering an effective amount of vector of claim 12 to the subject.

28.-32. (canceled)

33. A kit comprising one or more of the isolated polypeptide of claim 1, or an equivalent thereof, and instructions for use.


SEQUENCE LISTING (86 sequences)

SEQ ID NO: 1 - protein, 12 aa, Artificial Sequence
  Description of Artificial Sequence: Synthetic peptide
  MOD_RES (1)..(3): Any basic amino acid or not present
  MOD_RES (5)..(5): Any hydrophobic amino acid or not present
  MOD_RES (6)..(6): Ser, Ala, Pro or Gly
  MOD_RES (7)..(7): Any hydrophobic amino acid or not present
  MOD_RES (8)..(9): Any basic amino acid or not present
  MOD_RES (10)..(10): Ser, Ala, Pro or Gly
  MOD_RES (11)..(11): Any hydrophobic amino acid or not present
  MOD_RES (12)..(12): Any basic amino acid or not present
  See specification as filed for detailed description of substitutions and preferred embodiments
XXXRXXXXXXXX

SEQ ID NO: 2 - protein, 13 aa, Artificial Sequence
  Description of Artificial Sequence: Synthetic peptide
  MOD_RES (7)..(7): Ser, Ala, Pro or Gly
  MOD_RES (11)..(11): Ser, Ala, Pro or Gly
KKKKRFXFKKXFK

SEQ ID NO: 3 - protein, 25 aa, Artificial Sequence
  Description of Artificial Sequence: Synthetic peptide
  MOD_RES (8)..(8): Ser, Ala, Pro or Gly
  MOD_RES (12)..(12): Ser, Ala, Pro or Gly
  MOD_RES (16)..(16): Ser, Ala, Pro or Gly
  MOD_RES (19)..(19): Ser, Ala, Pro or Gly
KKKKKRFXFKKXFKLXGFXFKKNKK

SEQ ID NO: 4 - protein, 12 aa, Artificial Sequence
  Description of Artificial Sequence: Synthetic peptide
  MOD_RES (1)..(3): Any basic amino acid or not present
  MOD_RES (5)..(5): Any hydrophobic amino acid or not present
  MOD_RES (7)..(7): Any hydrophobic amino acid or not present
  MOD_RES (8)..(9): Any basic amino acid or not present
  MOD_RES (11)..(11): Any hydrophobic amino acid or not present
  MOD_RES (12)..(12): Any basic amino acid or not present
  See specification as filed for detailed description of substitutions and preferred embodiments
XXXRXSXXXSXX

SEQ ID NO: 5 - protein, 12 aa, Artificial Sequence
  Description of Artificial Sequence: Synthetic peptide
KKKRFSFKKSFK

SEQ ID NO: 6 - protein, 13 aa, Artificial Sequence
  Description of Artificial Sequence: Synthetic peptide
KKKKRFSFKKSFK

SEQ ID NO: 7 - protein, 12 aa, Artificial Sequence
  Description of Artificial Sequence: Synthetic peptide
  MOD_RES (5)..(5): Ala, Ile, Leu, Val, Trp or Tyr
  MOD_RES (7)..(7): Ala, Ile, Leu, Val, Trp or Tyr
  MOD_RES (11)..(11): Ala, Ile, Leu, Val, Trp or Tyr
KKKRXSXKKSXK

SEQ ID NO: 8 - protein, 25 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(22): Any basic amino acid or not present
  MOD_RES (24)..(25): Any basic amino acid or not present
  See specification as filed for detailed description of substitutions and preferred embodiments
XXXXXRXSXXXSXXLSGXSXXXNXX

SEQ ID NO: 9 - protein, 12 aa, Artificial Sequence
  Description of Artificial Sequence: Synthetic peptide
  MOD_RES (1)..(3): His or Arg
  MOD_RES (8)..(9): His or Arg
  MOD_RES (12)..(12): His or Arg
XXXRFSFXXSFX

SEQ ID NO: 10 - protein, 25 aa, Artificial Sequence
  Description of Artificial Sequence: Synthetic peptide
  MOD_RES (7)..(7): Ala, Ile, Leu, Val, Trp or Tyr
  MOD_RES (9)..(9): Ala, Ile, Leu, Val, Trp or Tyr
  MOD_RES (13)..(13): Ala, Ile, Leu, Val, Trp or Tyr
KKKKKRXSXKKSXKLSGFSFKKNKK

SEQ ID NO: 11 - protein, 25 aa, Artificial Sequence
  Description of Artificial Sequence: Synthetic peptide
  MOD_RES (1)..(5): His or Arg
  MOD_RES (10)..(11): His or Arg
  MOD_RES (14)..(14): His or Arg
  MOD_RES (21)..(22): His or Arg
  MOD_RES (24)..(25): His or Arg
XXXXXRFSFXXSFXLSGFSFXXNXX

SEQ ID NO: 12 - protein, 25 aa, Artificial Sequence
  Description of Artificial Sequence: Synthetic peptide
KKKKKRFSFKKSFKLSGFSFKKNKK

SEQ ID NO: 13 - protein, 25 aa, Artificial Sequence
  Description of Artificial Sequence: Synthetic peptide
KKKKKRFAFKKAFKLAGFAFKKNKK

SEQ ID NO: 14 - protein, 12 aa, Artificial Sequence
  Description of Artificial Sequence: Synthetic peptide
  MOD_RES (1)..(3): Any basic amino acid or not present
  MOD_RES (5)..(5): Any hydrophobic amino acid or not present
  MOD_RES (7)..(7): Any hydrophobic amino acid or not present
  MOD_RES (8)..(9): Any basic amino acid or not present
  MOD_RES (11)..(11): Any hydrophobic amino acid or not present
  MOD_RES (12)..(12): Any basic amino acid or not present
  See specification as filed for detailed description of substitutions and preferred embodiments
XXXRXAXXXAXX

SEQ ID NO: 15 - protein, 25 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(22): Any basic amino acid or not present
  MOD_RES (24)..(25): Any basic amino acid or not present
  See specification as filed for detailed description of substitutions and preferred embodiments
XXXXXRXAXXXAXXLAGXAXXXNXX

SEQ ID NO: 16 - protein, 12 aa, Artificial Sequence
  Description of Artificial Sequence: Synthetic peptide
KKKRFAFKKAFK

SEQ ID NO: 17-23 - skipped (7 entries)

SEQ ID NO: 24 - protein, 12 aa, Artificial Sequence
  Description of Artificial Sequence: Synthetic peptide
  MOD_RES (1)..(3): Any basic amino acid or not present
  MOD_RES (5)..(5): Any hydrophobic amino acid or not present
  MOD_RES (7)..(7): Any hydrophobic amino acid or not present
  MOD_RES (8)..(9): Any basic amino acid or not present
  MOD_RES (11)..(11): Any hydrophobic amino acid or not present
  MOD_RES (12)..(12): Any basic amino acid or not present
  See specification as filed for detailed description of substitutions and preferred embodiments
XXXRXSXXXSXX

SEQ ID NO: 25 - protein, 13 aa, Artificial Sequence
  Description of Artificial Sequence: Synthetic peptide
  MOD_RES (1)..(4): Any basic amino acid or not present
  MOD_RES (6)..(6): Any hydrophobic amino acid or not present
  MOD_RES (8)..(8): Any hydrophobic amino acid or not present
  MOD_RES (9)..(10): Any basic amino acid or not present
  MOD_RES (12)..(12): Any hydrophobic amino acid or not present
  MOD_RES (13)..(13): Any basic amino acid or not present
  See specification as filed for detailed description of substitutions and preferred embodiments
XXXXRXSXXXSXX

SEQ ID NO: 26 - protein, 14 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  See specification as filed for detailed description of substitutions and preferred embodiments
XXXXXRXSXXXSXX

SEQ ID NO: 27 - protein, 15 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  See specification as filed for detailed description of substitutions and preferred embodiments
XXXXXRXSXXXSXXL

SEQ ID NO: 28 - protein, 16 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  See specification as filed for detailed description of substitutions and preferred embodiments
XXXXXRXSXXXSXXLS

SEQ ID NO: 29 - protein, 17 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  See specification as filed for detailed description of substitutions and preferred embodiments
XXXXXRXSXXXSXXLSG

SEQ ID NO: 30 - protein, 18 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  See specification as filed for detailed description of substitutions and preferred embodiments
XXXXXRXSXXXSXXLSGX

SEQ ID NO: 31 - protein, 19 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  See specification as filed for detailed description of substitutions and preferred embodiments
XXXXXRXSXXXSXXLSGXS

SEQ ID NO: 32 - protein, 20 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  See specification as filed for detailed description of substitutions and preferred embodiments
XXXXXRXSXXXSXXLSGXSX

SEQ ID NO: 33 - protein, 21 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(21): Any basic amino acid or not present
  See specification as filed for detailed description of substitutions and preferred embodiments
XXXXXRXSXXXSXXLSGXSXX

SEQ ID NO: 34 - protein, 22 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(22): Any basic amino acid or not present
  See specification as filed for detailed description of substitutions and preferred embodiments
XXXXXRXSXXXSXXLSGXSXXX

SEQ ID NO: 35 - protein, 23 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(22): Any basic amino acid or not present
  See specification as filed for detailed description of substitutions and preferred embodiments
XXXXXRXSXXXSXXLSGXSXXXN

SEQ ID NO: 36 - protein, 24 aa, Artificial Sequence
  Description of Artificial Sequence: Synthetic peptide
  MOD_RES (1)..(5): Any basic amino acid or not present
  MOD_RES (7)..(7): Any hydrophobic amino acid or not present
  MOD_RES (9)..(9): Any hydrophobic amino acid or not present
  MOD_RES (10)..(11): Any basic amino acid or not present
  MOD_RES (13)..(13): Any hydrophobic amino acid or not present
  MOD_RES (14)..(14): Any basic amino acid or not present
  MOD_RES (18)..(18): Any hydrophobic amino acid or not present
  MOD_RES (20)..(20): Any hydrophobic amino acid or not present
  MOD_RES (21)..(22): Any basic amino acid or not present
  MOD_RES (24)..(24): Any basic amino acid or not present
  See specification as filed for detailed description of substitutions and preferred embodiments
XXXXXRXSXXXSXXLSGXSXXXNX

SEQ ID NO: 37 - protein, 25 aa, Artificial Sequence
  Description of Artificial Sequence: Synthetic peptide
KKKKKRFSFKKSFKLSGFSFKKNKK

SEQ ID NO: 38 - protein, 25 aa, Artificial Sequence
  Description of Artificial Sequence: Synthetic peptide
KKKKKRFDFKKDFKLDGFDFKKNKK

SEQ ID NO: 39 - protein, 12 aa, Artificial Sequence
  Description of Artificial Sequence: Synthetic peptide
  MOD_RES (1)..(3): Any basic amino acid or not present
  MOD_RES (5)..(5): Any hydrophobic amino acid or not present
  MOD_RES (6)..(6): Ser, Ala, Pro or Gly
  MOD_RES (7)..(7): Any hydrophobic amino acid or not present
  MOD_RES (8)..(9): Any basic amino acid or not present
  MOD_RES (10)..(10): Ser, Ala, Pro or Gly
  MOD_RES (11)..(11): Any hydrophobic amino acid or not present
  MOD_RES (12)..(12): Any basic amino acid or not present
  See specification as filed for detailed description of substitutions and preferred embodiments
XXXRXXXXXXXX

SEQ ID NO: 40 - protein, 20 aa, Artificial Sequence
  Description of Artificial Sequence: Synthetic peptide
FSFGSFSLKKFSFRKKKNKK

SEQ ID NO: 41 - protein, 24 aa, Artificial Sequence
  Description of Artificial Sequence: Synthetic peptide
KKKKFSFGSFSLKKFSFRKKKNKK

SEQ ID NO: 42 - protein, 24 aa, Artificial Sequence
  Description of Artificial Sequence: Synthetic peptide
KKKKFAFGAFALKKFAFRKKKNKK

SEQ ID NO: 43 - protein, 20 aa, Artificial Sequence
  Description of Artificial Sequence: Synthetic peptide
KKKNKSFFGKSKKFKKKKSF

SEQ ID NO: 44 - protein, 25 aa, Artificial Sequence
  Description of Artificial Sequence: Synthetic peptide
KRFLSKKKNKSFFGKSKKFKKKKSF

SEQ ID NO: 45 - protein, 25 aa, Artificial Sequence
  Description of Artificial Sequence: Synthetic peptide
  MOD_RES (6)..(7): D-amino acid
  MOD_RES (9)..(10): D-amino acid
  MOD_RES (13)..(15): D-amino acid
  MOD_RES (18)..(21): D-amino acid
  MOD_RES (24)..(24): D-amino acid
KKKKKRFAFKKAFKLAGFAFKKNKK

SEQ ID NO: 46 - protein, 25 aa, Artificial Sequence
  Description of Artificial Sequence: Synthetic peptide
  MOD_RES (1)..(2): D-amino acid
  MOD_RES (24)..(25): D-amino acid
KKKKKRFAFKKAFKLAGFAFKKNKK

SEQ ID NO: 47 - protein, 25 aa, Artificial Sequence
  Description of Artificial Sequence: Synthetic peptide
  MOD_RES (6)..(6): D-amino acid
  MOD_RES (8)..(8): D-amino acid
  MOD_RES (10)..(13): D-amino acid
  MOD_RES (15)..(16): D-amino acid
  MOD_RES (18)..(20): D-amino acid
  MOD_RES (24)..(24): D-amino acid
KKKKKFAFGAFALKKFAFRKKKNKK

SEQ ID NO: 48 - protein, 25 aa, Artificial Sequence
  Description of Artificial Sequence: Synthetic peptide
KKKKKRFSFKKSFKLSGFSFKANKK

SEQ ID NO: 49 - protein, 25 aa, Artificial Sequence
  Description of Artificial Sequence: Synthetic peptide
KKKKKRFSFKASFKLSGFSFKKNKK

SEQ ID NO: 50 - protein, 25 aa, Artificial Sequence
  Description of Artificial Sequence: Synthetic peptide
KKKKKRFSFAKSFKLSGFSFKKNKK

SEQ ID NO: 51 - protein, 25 aa, Artificial Sequence
  Description of Artificial Sequence: Synthetic peptide
KKKKKAFSFKKSFKLSGFSFKKNKK

SEQ ID NO: 52 - protein, 25 aa, Artificial Sequence
  Description of Artificial Sequence: Synthetic peptide
KKAKKRFSFKKSFKLSGFSFKKNKK

SEQ ID NO: 53 - protein, 25 aa, Artificial Sequence
  Description of Artificial Sequence: Synthetic peptide
AKKKKRFSFKKSFKLSGFSFKKNKK

SEQ ID NO: 54 - protein, 25 aa, Artificial Sequence
  Description of Artificial Sequence: Synthetic peptide
KKKKKRFSFKKSFKLSGFSFKKNKK

SEQ ID NO: 55 - protein, 7 aa, Artificial Sequence
  Description of Artificial Sequence: Synthetic peptide
  MOD_RES (1)..(1): Any amino acid
  MOD_RES (2)..(2): Lys, Arg or Ala
  MOD_RES (4)..(4): Ala or Ser
  MOD_RES (7)..(7): Any amino acid
XXFXFRX

SEQ ID NO: 56 - protein, 7 aa, Artificial Sequence
  Description of Artificial Sequence: Synthetic peptide
  MOD_RES (1)..(1): Any amino acid
  MOD_RES (2)..(2): Lys, Arg or Ala
  MOD_RES (4)..(4): Ala or Ser
  MOD_RES (7)..(7): Any amino acid
  See specification as filed for detailed description of substitutions and preferred embodiments
XXFXFRX

SEQ ID NO: 57 - protein, 25 aa, Artificial Sequence
  Description of Artificial Sequence: Synthetic peptide
KKKKKRFSFKKSFKLSGFSFKKNKK

SEQ ID NO: 58 - protein, 12 aa, Artificial Sequence
  Description of Artificial Sequence: Synthetic peptide
  MOD_RES (1)..(3): Any amino acid
  MOD_RES (5)..(5): Any hydrophobic amino acid
  MOD_RES (7)..(7): Any hydrophobic amino acid
  MOD_RES (8)..(9): Any amino acid
  MOD_RES (11)..(11): Any hydrophobic amino acid
  MOD_RES (12)..(12): Any amino acid
XXXRYAYXXAYX

SEQ ID NO: 59 - protein, 25 aa, Artificial Sequence
  Description of Artificial Sequence: Synthetic peptide
  MOD_RES (1)..(5): Any amino acid
  MOD_RES (7)..(7): Any hydrophobic amino acid
  MOD_RES (9)..(9): Any hydrophobic amino acid
  MOD_RES (10)..(11): Any amino acid
  MOD_RES (13)..(13): Any hydrophobic amino acid
  MOD_RES (14)..(14): Any amino acid
  MOD_RES (18)..(18): Any hydrophobic amino acid
  MOD_RES (20)..(20): Any hydrophobic amino acid
  MOD_RES (21)..(22): Any amino acid
  MOD_RES (24)..(25): Any amino acid
XXXXXRYAYXXAYXLAGYAYXXNXX

SEQ ID NO: 60 - DNA, 20 bp, Artificial Sequence
  Description of Artificial Sequence: Synthetic primer
tcctcatcctcccttgagaa

SEQ ID NO: 61 - DNA, 20 bp, Artificial Sequence
  Description of Artificial Sequence: Synthetic primer
atgaaggatggctggaacag

SEQ ID NO: 62 - DNA, 24 bp, Artificial Sequence
  Description of Artificial Sequence: Synthetic primer
acgaagacatcccaccaatcacct

SEQ ID NO: 63 - DNA, 24 bp, Artificial Sequence
  Description of Artificial Sequence: Synthetic primer
agatcacgtcatcgcacaacacct

SEQ ID NO: 64 - DNA, 18 bp, Artificial Sequence
  Description of Artificial Sequence: Synthetic primer
agagacttggatgaggag

SEQ ID NO: 65 - DNA, 18 bp, Artificial Sequence
  Description of Artificial Sequence: Synthetic primer
ctgagaatgctggagatg

SEQ ID NO: 66 - DNA, 20 bp, Artificial Sequence
  Description of Artificial Sequence: Synthetic primer
tccacaagcgtcatgaagag

SEQ ID NO: 67 - DNA, 20 bp, Artificial Sequence
  Description of Artificial Sequence: Synthetic primer
ctctgaatcctggcattggt

SEQ ID NO: 68 - DNA, 22 bp, Artificial Sequence
  Description of Artificial Sequence: Synthetic primer
aacttctcagcatcacgatgac

SEQ ID NO: 69 - DNA, 23 bp, Artificial Sequence
  Description of Artificial Sequence: Synthetic primer
ttgtaggagtgtcggttgttaag

SEQ ID NO: 70 - DNA, 24 bp, Artificial Sequence
  Description of Artificial Sequence: Synthetic primer
ttgttgaagaagccagcatgggtg

SEQ ID NO: 71 - DNA, 24 bp, Artificial Sequence
  Description of Artificial Sequence: Synthetic primer
ttaccttcacgtggccattctcct

SEQ ID NO: 72 - DNA, 19 bp, Artificial Sequence
  Description of Artificial Sequence: Synthetic oligonucleotide
gagaaggcggtgaggctga

SEQ ID NO: 73 - DNA, 19 bp, Artificial Sequence
  Description of Artificial Sequence: Synthetic oligonucleotide
tcagcctcaccgccttctc

SEQ ID NO: 74 - DNA, 19 bp, Artificial Sequence
  Description of Artificial Sequence: Synthetic oligonucleotide
gaaggtaaacggcgacgct

SEQ ID NO: 75 - DNA, 19 bp, Artificial Sequence
  Description of Artificial Sequence: Synthetic oligonucleotide
agcgtcgccgtttaccttc

SEQ ID NO: 76 - DNA, 20 bp, Artificial Sequence
  Description of Artificial Sequence: Synthetic oligonucleotide
gagcgcttctccttcaagaa

SEQ ID NO: 77 - DNA, 20 bp, Artificial Sequence
  Description of Artificial Sequence: Synthetic oligonucleotide
ttcttgaaggagaagcgctc

SEQ ID NO: 78 - DNA, 34 bp, Artificial Sequence
  Description of Artificial Sequence: Synthetic primer
gatccatgggtgcccagttctccaagaccgcagc

SEQ ID NO: 79 - DNA, 33 bp, Artificial Sequence
  Description of Artificial Sequence: Synthetic primer
tctagactctctgccgcctccgctgggggggct

SEQ ID NO: 80 - DNA, 35 bp, Artificial Sequence
  Description of Artificial Sequence: Synthetic primer
gaagcgctttgccttcaagaagtctttcaagctga

SEQ ID NO: 81 - DNA, 34 bp, Artificial Sequence
  Description of Artificial Sequence: Synthetic primer
tcagcttgaaagacttcttgaagcaaagcgcttc

SEQ ID NO: 82 - DNA, 34 bp, Artificial Sequence
  Description of Artificial Sequence: Synthetic primer
gaagccttttccttcaagaaggctttcaagctga

SEQ ID NO: 83 - DNA, 35 bp, Artificial Sequence
  Description of Artificial Sequence: Synthetic primer
tcagcttgaaagccttcttgaaggaaaagcgcttc

SEQ ID NO: 84 - protein, 12 aa, Artificial Sequence
  Description of Artificial Sequence: Synthetic peptide
  MOD_RES (4)..(7): Any amino acid
  MOD_RES (10)..(11): Any amino acid
KKKXXXXKKXXK

SEQ ID NO: 85 - protein, 15 aa, Artificial Sequence
  Description of Artificial Sequence: Synthetic peptide
PSPSNETPKKKKKRF

SEQ ID NO: 86 - protein, 8 aa, Artificial Sequence
  Description of Artificial Sequence: Synthetic peptide
NGQENGHV